(19) United States
(12) Patent Application Publication
(10) Pub. No.: US 2019/0328900 A1
(43) Pub. Date: Oct. 31, 2019

(54) ANTIBODY-DRUG CONJUGATES AND THERAPEUTIC METHODS USING THE SAME

(71) Applicant: GLAXOSMITHKLINE INTELLECTUAL PROPERTY DEVELOPMENT LIMITED, Brentford, Middlesex (GB)

(72) Inventors: Jerry JEFFREY, Research Triangle Park, NC (US); Jun TANG, Research Triangle Park, NC (US); Vincent Wing-Fai TAI, Research Triangle Park, NC (US); David TEMELKOFF, Research Triangle Park, NC (US); Emile Johann VELTHUISEN, Research Triangle Park, NC (US); Jason Gordon WEATHERHEAD, Research Triangle Park, NC (US)

(21) Appl. No.: 16/308,862

(22) PCT Filed: Jun. 30, 2017
(86) PCT No.: PCT/IB2017/053979

§ 371 Date: Dec. 11, 2018

Related U.S. Application Data
(60) Provisional application No. 62/357,410, filed on Jul. 1, 2016.

(51) Int. Cl. A61K 47/68; A61P 31/18; C07K 16/10; A61K 47/65; A61K 31/4985

(52) U.S. Cl. CPC A61K 47/6841; A61P 31/18; A61K 31/4985; C07K 16/1063; A61K 47/65; A61K 47/6849

(57) ABSTRACT

The invention discloses an antibody-drug conjugate of Formula (I):

Ab-[L-Dₙ]ₓ (I)
wherein:
Ab comprises a broadly neutralizing anti-HIV antibody;
L comprises a linker molecule covalently bonded to said broadly neutralizing anti-HIV antibody;
D comprises one or more drugs comprising an HIV therapeutic compound covalently bonded to said linker molecule L, wherein said one or more broadly neutralizing anti-HIV antibodies Ab specifically bind to an HIV envelope glycoprotein and said one or more drugs D specifically bind to an HIV envelope glycoprotein;
n is selected from 1-4; and
x is selected from 1-12.

The instant application claims priority to U.S. Provisional Patent Application Ser. No. 62/357,410 filed Jul. 1, 2016. The content of this application is incorporated by reference herein in its entirety.

### SEQUENCE LISTING

The instant application contains a Sequence Listing which has been filed electronically in ASCII format and is hereby incorporated by reference in its entirety. Said ASCII copy, created on Jun. 29, 2017, is named PR66117_SL.txt and is 37,133 bytes in size.

### FIELD OF THE INVENTION

The present invention relates to antibody-drug conjugates, pharmaceutical compositions, and methods of use thereof in connection with individuals infected with HIV.

### BACKGROUND OF THE INVENTION

The human immunodeficiency virus (HIV types1 and 2) leads to the contraction of acquired immune deficiency disease (AIDS). Unfortunately, the number of cases of HIV continues to rise, and currently over twenty-five million individuals worldwide suffer from the virus. Presently, long-term suppression of viral replication with antiretroviral drugs is the only option for treating an HIV infection. Indeed, the U.S. Food and Drug Administration has approved twenty-five drugs over six different inhibitor classes, which have been shown to greatly increase patient survival and quality of life. However, additional therapies are still required due to a number of issues including, but not limited to, undesirable drug-drug interactions; drug-food interactions; non-adherence to therapy; drug resistance due to mutation of the viral target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV variants. Despite the positive impact of HAART on patient survival, drug resistance can still occur and the survival and quality of life are not normalized as compared to uninfected persons [Lohse Ann Intern Med 2007 146; 87-95]. Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155]. This increased incidence of non-AIDS morbidity/mortality occurs in the context of, and is potentially caused by, elevated systemic inflammation related to the immunologic damage caused by HIV infection [Hunt J Infect Dis 2014][Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within most immune cells in the infected individual and may reactivate at any time, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013][Henrich T J Ann Intern Med 2014]. In one case, the viral reservoir was eliminated during treatment of leukemia and no viral rebound was observed during several years of follow-up [Hutter G N Engl J Med 2009]. These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. Induction of the latent reservoir typically results in either direct death of the latently infected cell or killing of the induced cell by the immune system after the virus is made visible. As this is performed during ART, viral genomes produced are believed to not result in the infection of new cells and the size of the reservoir may decay.

Despite the success of HAART, the virus ultimately generates resistance over time perpetuating the need for future ARTs. In addition to xenobiotic treatment of HIV, the immune system produces antibodies to HIV during the course of infection primarily targeted to the HIV envelope protein, gp160. These antibodies bind to the virion and neutralize the ability of the virion to infect additional target cells. Recent technologies have provided platforms to isolate neutralizing antibodies from infected individuals and over time better antibodies have been discovered that neutralize diverse sequences of gp160. Various broadly neutralizing antibodies (bNAbs) are being explored as ARTs by infusion into HIV infected individuals or relevant models. Such bnAbs may also address issues such as patient compliance due to their longer circulating half-life compared to historical ART small molecules and could result in once monthly or even longer dosing regimens.

In view of the above, there is a continuing need in the art to develop additional therapeutic approaches for treating HIV infected individuals and to address such issues as patient compliance and potential reduction of ART dosing frequency. Moreover, there is a need to employ improved means to target gp160 to attempt to increase the breadth of gp160 diversity inhibited and improve durability by providing multiple anti-viral targets in one agent analogous to HAART provided by multiple small molecules.

### SUMMARY OF THE INVENTION

In one aspect, the invention provides an antibody-drug conjugate of Formula (I):

Ab-L-D (I)

wherein:

Ab comprises a broadly neutralizing antibody;

L comprises a linker molecule covalently bonded to said broadly neutralizing antibody; and

D comprises one or more drugs covalently bonded to said linker molecule, said one or more drugs specifically bind to said HIV envelope glycoprotein.

In another aspect, the invention provides an antibody-drug conjugate of Formula (II):

Ab[L-Dₙ]ₓ (II)

wherein:

Ab comprises a broadly neutralizing anti-HIV antibody;

L comprises a linker molecule covalently bonded to said broadly neutralizing anti-HIV antibody;

D comprises one or more drugs comprising an HIV attachment inhibitor compound covalently bonded to said linker molecule, wherein said one or more broadly neutralizing anti-HIV antibodies specifically bind to an HIV envelope glycoprotein;

n is selected from 1-4; and

x is selected from 1-12.

Also provided are pharmaceutical compositions comprising the antibody-drug conjugate of Formulas (I) and (II) and methods of treating HIV infected patients with the antibody-drug conjugate of Formula (I) and (II).

These and other aspects are encompassed by the invention as set forth herein.

### BRIEF DESCRIPTION OF THE DRAWINGS

FIG. 1 illustrates size exclusion chromatography (SEC-HPLC) analysis for the broadly neutralizing antibody VRC01;

FIG. 2 illustrates sodium dodecyl sulfate polyacrylamide gel electrophoresis (SDS-PAGE) for the broadly neutralizing antibody VRC01.

FIGS. 3A and 3B illustrate structures for antibody-drug conjugates of the invention;

FIGS. 4A and 4B illustrate structures for antibody-drug conjugates of the invention; and

FIGS. 5A and 5B illustrate structures for antibody-drug conjugates of the invention;

FIG. 6 illustrates the structure of a drug-linker for use with an antibody-drug-conjugate;

FIG. 7 illustrates the structure of a drug for use with an antibody-drug-conjugate;

FIG. 8 illustrates the structure of a drug-linker for use with an antibody-drug-conjugate;

FIG. 9 illustrates the structure of a drug for use with an antibody-drug-conjugate; and

FIG. 10 illustrates the structure of a surrogate compound of gp160 attachment inhibitor-linker.

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

All issued patents, published patent applications and other publications as referenced herein are deemed to be incorporated herein by reference in their entirety.

In one aspect, the invention provides an antibody-drug conjugate of Formula (I):

Ab-L-D (I)

wherein:

Ab comprises a broadly neutralizing antibody;

L comprises a linker molecule covalently bonded to said broadly neutralizing antibody; and

D comprises one or more drugs covalently bonded to said linker molecule, wherein said one or more drugs specifically bind to said HIV envelope glycoprotein.

In another aspect, the invention provides an antibody-drug conjugate of Formula (II):

Ab-[L-Dₙ]ₓ (II)

wherein:

Ab comprises a broadly neutralizing anti-HIV antibody;

L comprises a linker molecule covalently bonded to said broadly neutralizing anti-HIV antibody;

D comprises one or more drugs comprising an HIV attachment inhibitor compound covalently bonded to said linker molecule, wherein said one or more broadly neutralizing anti-HIV antibodies specifically bind to an HIV envelope glycoprotein;

n is selected from 1-4; and

x is selected from 1-12.

In yet another aspect, the invention provides an antibody-drug conjugate of Formula (II):

Ab-[L-Dₙ]ₓ (II)

wherein:

Ab comprises a broadly neutralizing anti-HIV antibody;

L comprises a linker molecule covalently bonded to said broadly neutralizing anti-HIV antibody;

D comprises one or more drugs comprising an HIV attachment inhibitor compound covalently bonded to said linker molecule, wherein said one or more broadly neutralizing anti-HIV antibodies specifically bind to an HIV envelope glycoprotein;

n is selected from 1-2; and

x is selected from 2-4.

In yet another aspect, the invention provides an antibody-drug conjugate of Formula (II):

Ab-[L-Dₙ]ₓ (II)

wherein:

Ab comprises a broadly neutralizing anti-HIV antibody;

L comprises a linker molecule covalently bonded to said broadly neutralizing anti-HIV antibody;

D comprises one or more drugs comprising an HIV attachment inhibitor compound covalently bonded to said linker molecule, wherein said one or more broadly neutralizing anti-HIV antibodies specifically bind to an HIV envelope glycoprotein;

n is 1; and

x is 2.

In another aspect, the invention provides an antibody-drug conjugate of the Formula (I):

Ab-L-D (I)

wherein:

Ab comprises a broadly neutralizing antibody having a binding affinity for an HIV envelope glycoprotein;

L comprises one or more linkers molecule covalently bonded to said broadly neutralizing antibody; and

D comprises one or more drugs covalently bonded to said one or more linker molecules, said one or more drugs capable of binding to said HIV envelope glycoprotein.

In another aspect, the invention provides an antibody-drug conjugate of Formula (I):

Ab-[L-Dₙ]ₓ (I)

wherein:

Ab comprises a broadly neutralizing anti-HIV antibody;

L comprises a linker molecule covalently bonded to said broadly neutralizing anti-HIV antibody;

D comprises one or more drugs comprising an HIV therapeutic compound covalently bonded to said linker molecule L, wherein said one or more broadly neutralizing anti-HIV antibodies Ab specifically bind to an HIV envelope glycoprotein and said one or more drugs D specifically bind to an HIV envelope glycoprotein;

n is selected from 1-4; and

x is selected from 1-12.

Preferably, n is selected from 1-2; and

x is selected from 2-4.

More preferably, n is 1; and

x is 1 or 2.

In another aspect, the invention provides an antibody-drug conjugate of Formula (I):

Ab-[L-Dₙ]ₓ (I)

wherein:

Ab comprises a broadly neutralizing anti-HIV antibody;

L comprises a linker molecule covalently bonded to said broadly neutralizing anti-HIV antibody;

D comprises one or more drugs comprising an HIV therapeutic compound covalently bonded to said linker molecule L, wherein said one or more broadly neutralizing anti-HIV antibodies Ab specifically bind to an HIV envelope glycoprotein and said one or more drugs D specifically bind to an HIV envelope glycoprotein;

n is selected from 1-4;

x is selected from 1-12, wherein the antibody-drug-conjugate comprises (1) a first drug D covalently bonded to a first linker molecule L, which is covalently bonded to said broadly neutralizing antibody and (2) a second drug D covalently bonded to a second linker molecule L, which is covalently bonded to said broadly neutralizing antibody.

In one embodiment, the first drug D is the same as the second drug D.

In one embodiment, the first drug D is different than the second drug D.

In one embodiment, the first linker and the second linker may be the same or different. In one embodiment, the first drug and the first linker are attached to the broadly neutralizing antibody at a different location than the second drug and second linker.

An “antibody” is defined as a polypeptide including at least a light chain or heavy chain immunoglobulin variable region which specifically recognizes and binds an epitope of an antigen, or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. The term antibody includes intact immunoglobulins, as well the variants and portions thereof, such as a single variable domain (e.g., VH, VHH, VL, domain antibody (DAB)), Fab′ fragments, F(ab)′₂ fragments, single chain Fv proteins (“scFv”), disulfide stabilized Fv proteins (“dsFv”), diabodies, TANDABS etc. and modified versions of any of the foregoing. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “DAB”” may be considered the same as a “single variable domain”. A single variable domain may be a human single variable domain, but also includes single variable domains from other species such as rodent nurse shark and Camelid VHH DABS. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains.

Typically, a naturally occurring immunoglobulin has heavy (H) chains and light (L) chains interconnected by disulfide bonds. There are two types of light chain, lambda (A) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarily-determining regions” or “CDRs”. The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3, otherwise known as CDRH3, is the CDR3 located in the variable domain of the heavy chain of the antibody in which it is found, whereas a V_{L} CDR1, otherwise known as CDRL1, is the CDR1 from the variable domain of the light chain of the antibody in which it is found. An antibody that binds a target protein will have a specific V_{H} region and the V_{L} region sequence, and thus specific CDR sequences. Antibodies with different specificities (such as different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3”, and unless otherwise noted, follow the Kabat numbering convention. It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person. Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | | | | | Minimum |
|---|---|---|---|---|---|
| | Kabat | Chothia | AbM | Contact | binding |
| | CDR | CDR | CDR | CDR | unit |
| H1 | 31-35/35A/ | 26-32/33/ | 26-35/35A/ | 30-35/35A/ | 31-32 |
| | 35B | 34 | 35B | 35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

References to “V_{H}” or “V_{H}” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab. An antibody or other active agent “binds to (e.g., specifically),” is “specific to/for” or “recognizes” (e.g., specifically) an antigen if such is able to discriminate between the antigen and one or more reference antigen(s), since binding specificity is not an absolute, but a relative property. In its most general form (and when no defined reference is mentioned), “binding” is referring to the ability of the antibody or active agent to discriminate between the antigen of interest and an unrelated antigen, as may be determined, for example, in accordance with one of the following methods. Such methods comprise, but are not limited to Western blots, ELISA-, RIA-, ECL-, IRMA-tests and peptide scans. The scoring may be carried out by standard color development (e.g. secondary antibody with horseradish peroxide and tetramethyl benzidine with hydrogenperoxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like. Additionally, “binding”, and more particularly “specific binding” may refer to the ability of an antibody to discriminate between the target antigen and one or more closely related antigen(s), which are used as reference points. Additionally, “binding” may relate to the ability of an antibody to discriminate between different parts of its target antigen, e.g. different domains or regions, or between one or more key amino acid residues or stretches of amino acid residues.

“Affinity” or “binding affinity” refers to e.g., the strength of the sum total of non-covalent interactions between a single binding site of an active agent (e.g. an antibody or molecule) and its binding partner (e.g. an antigen). Unless indicated otherwise, as used herein, “binding affinity” refers to intrinsic binding affinity which reflects a 1:1 interaction between members of a binding pair (e.g. antibody and antigen). Affinity can be measured by common methods known in the art, including equilibrium methods (e.g. enzyme-linked immunoabsorbent assay (ELISA) or radioimmunoassay (RIA)), or kinetics (e.g. BIACORE analysis). A particular method for measuring affinity is Surface Plasmon Resonance (SPR).

For example with respect to the term “binding affinity”, under designated conditions, an antibody that binds preferentially to a particular target protein (such as, e.g. gp120 or gp160) and does not bind in a significant amount to other proteins or polysaccharides present in the sample or subject, is referred to an antibody that specifically binds to its target. In one embodiment, affinity is calculated by a modification of the Scatchard method described by Frankel et al., Mol. Immunol., 16: 101-106, 1979. In another embodiment, binding affinity is measured by an antigen/antibody dissociation rate. In yet another embodiment, a binding affinity is measured by a competition radioimmunoassay. In several examples, a high binding affinity may range from about 1×10⁻⁶ M to about 1×10⁻¹² M, and more preferably from about 1×10⁻⁸ M to about 1×10⁻¹²M. (10 nM to 1 pM) (see e.g., WO 2012/106578)

“Avidity” is the sum total of the strength of binding of two molecules to one another at multiple sites, e.g. taking into account the valency of the interaction.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDR(s). Alternatively, the % identity may exclude the CDR(s), for example the CDR(s) is 100% identical to the subject sequence and the % identity variation is in the remaining portion of the query sequence, so that the CDR sequence is fixed/intact.

The VH or VL sequence may be a variant sequence with up to 10 amino acid substitutions, additions or deletions. For example, the variant sequence may have up to 9, 8, 7, 6, 5, 4, 3, 2 or 1 amino acid substitution(s), addition(s) or deletion(s).

The sequence variation may exclude the CDR(s), for example the CDR(s) is the same as the VH or VL (or HC or LC) sequence and the variation is in the remaining portion of the VH or VL (or HC or LC) sequence, so that the CDR sequence is fixed/intact.

In several embodiments, the constant region of the antibody includes one or more amino acid substitutions to optimize in vivo half-life of the antibody. The serum half-life of IgG Abs may be regulated by the neonatal Fe receptor (FcRn). Thus, in several embodiments, the antibody includes an amino acid substitution that increases binding to the FcRn. Several such substitutions are known to the person of ordinary skill in the art, such as substitutions at IgG constant regions T250Q and M428L (see, e.g. Hinton et al., J Immunol., 176:346-356, 2006); M428L and N434S (the “LS” mutation, see, e.g., Zalevsky, et al., Nature Biotechnology, 28:157-159, 2010); N434A (see, e.g., Petkova et al., Int. Immunol., 18: 1759-1769, 2006); T307 A, E380A, and N434A (see, e.g., Petkova et al., Int. Immunol., 18:1759-1769, 2006); and M252Y, S254T, and T256E (see, e.g., Dail′ Acqua et al., J. Biol. Chem., 281:23514-23524, 2006). The disclosed antibodies can comprise a Fc polypeptide including any of the substitutions listed above, for example, the Fc polypeptide can include the M428L and N434. As discussed, antibodies in accordance with the disclosure can be adapted or modified to provide increased serum half-life in vivo and consequently longer persistence, or residence, times of the functional activity of the antibody in the body. Suitably, such modified molecules have a decreased clearance and increased Mean Residence Time compared to the non-adapted molecule. Increased half-life can improve the pharmacokinetic and pharmacodynamic properties of a therapeutic molecule and can also be important for improved patient compliance.

Other suitable half-life extension strategies include: PEGylation, polysialylation, HESylation, recombinant PEG mimetics, N-glycosylation, O-glycosylation, Fc fusion, engineered Fc, IgG binding, albumin fusion, albumin binding, albumin coupling and nanoparticles.

Not intending to be bound by theory, the long half-life of IgG antibodies is reported to be dependent on its binding to FcRn. Therefore, substitutions that increase the binding affinity of IgG to FcRn at pH 6.0 while maintaining the pH dependence of the interaction by engineering the constant region have been studied (KUO, T. T. & AVESON, V. G. 2011. Neonatal Fc receptor and IgG-based therapeutics. MAbs, 3, 422-30)

In adult mammals, FcRn, also known as the neonatal Fc receptor, is capable of playing a key role in maintaining serum antibody levels by acting as a protective receptor that binds and salvages antibodies of the IgG isotype from degradation. IgG molecules are endocytosed by endothelial cells, and if they bind to FcRn, are recycled out into circulation. In contrast, IgG molecules that do not bind to FcRn enter the cells and are targeted to the lysosomal pathway where they are degraded.

The neonatal FcRn receptor is believed to be involved in both antibody clearance and the transcytosis across tissues, Kuo and Aveson, (2011). Human IgG1 residues that may interact with human FcRn includes Ile253, Ser254, Lys288, Thr307, GIn311, Asn434 and His435. Switches at any of these positions described in this section may enable increased serum half-life and/or altered effector properties of antibodies of the invention.

Antibodies suitable for use in the methods of the present invention as described herein may have amino acid modifications that may increase the affinity of the constant domain or fragment thereof for FcRn. Increasing the half-life of therapeutic and diagnostic IgG polypeptides and other bioactive molecules is capable of providing benefits e.g., including reducing the amount and/or frequency of dosing of these molecules. In one embodiment there is therefore provided an antibody according to the invention comprising all or a portion (an FcRn binding portion) of an IgG constant domain having one or more amino acid modifications.

A number of methods are known that can result in increased half-life (Kuo and Aveson, (2011)), including amino acid modifications that may be generated through techniques including alanine scanning mutagenesis, random mutagenesis and screening to assess the binding to FcRn and/or the in vivo behaviour. Computational strategies followed by mutagenesis may also be used to select one of amino acid mutations to mutate. Although substitutions in the constant region are able to improve the functions of therapeutic IgG antibodies, substitutions in the strictly conserved constant region may have the potential risk of immunogenicity in humans and substitution in the highly diverse variable region sequence might be less immunogenic. Reports concerned with the variable region include engineering the CDR residues to improve binding affinity to the antigen and engineering, the CDR and framework residues to improve stability and decrease immunogenicity risk. Improved affinity to the antigen may be achieved by affinity maturation using the phage or ribosome display of a randomized library.

Improved stability may be potentially obtained from sequence- and structure-based rational design. Decreased immunogenicity risk (deimmunization) can be accomplished by various humanization methodologies and the removal of T-cell epitopes, which can be predicted using in silico technologies or determined by in vitro assays. Additionally, variable regions have been engineered to lower pl. A longer half life was observed for these antibodies as compared to wild type antibodies despite comparable FcRn binding. Engineering or selecting antibodies with pH dependent antigen binding to modify antibody and/or antigen half-life e.g. IgG2 antibody half-life can be shortened if antigen-mediated clearance mechanisms normally degrade the antibody when bound to the antigen. Similarly, the antigen: antibody complex can impact the half-life of the antigen, either extending half-life by protecting the antigen from the typical degradation processes, or shortening the half-life via antibody-mediated degradation.

It may be appreciated that, upon production of antibody, in particular depending on the cell line used and particular amino acid sequence of the antigen binding protein, post-translational modifications may occur. For example, this may include the cleavage of certain leader sequences, the addition of various sugar moieties in various glycosylation and phosphorylation patterns, deamidation, oxidation, disulfide bond scrambling, isomerization, C-terminal lysine clipping, and N-terminal glutamine cyclisation. The present invention encompasses the use of antigen binding proteins which have been subjected to, or have undergone, one or more post-translational modifications. Thus an “antibody” of the invention includes an “antibody” as defined earlier which has undergone a post-translational modification such as described herein.

Deamidation is an enzymatic reaction primarily converting asparagine (N) to iso-aspartic acid (iso-aspartate) and aspartic acid (aspartate) (D) at approximately 3:1 ratio. This deamidation reaction is therefore related to isomerization of aspartate (D) to iso-aspartate.

The deamidation of asparagine and the isomerization of aspartate, both involve the intermediate succinimide. To a much lesser degree, deamidation can occur with glutamine residues in a similar manner. Deamidation can occur in a CDR, in a Fab (non-CDR region), or in the Fc region.

Oxidation can occur during production and storage (i.e. in the presence of oxidizing conditions) and results in a covalent modification of a protein, induced either directly by reactive oxygen species or indirectly by reaction with secondary by-products of oxidative stress. Oxidation happens primarily with methionine residues, but may occur at tryptophan and free cysteine residues. Oxidation can occur in a CDR, in a Fab (non-CDR) region, or in the Fc region.

Disulfide bond scrambling can occur during production and basic storage conditions. Under certain circumstances, disulfide bonds can break or form incorrectly, resulting in unpaired cysteine residues (—SH). These free (unpaired) sulfhydryls (—SH) can promote shuffling.

N-terminal glutamine (Q) and glutamate (glutamic acid) (E) in the heavy chain and/or light chain is likely to form pyroglutamate (pGlu) via cyclization. Most pGlu formation happens in the production bioreactor, but it can be formed non-enzymatically, depending on pH and temperature of processing and storage conditions. Cyclization of N-terminal Q or E is commonly observed in natural human antibodies.

C-terminal lysine clipping is an enzymatic reaction catalyzed by carboxypeptidases, and is commonly observed in recombinant and natural human antibodies. Variants of this process include removal of lysine from one or both heavy chains due to cellular enzymes from the recombinant host cell. Upon administration to the human subject/patient is likely to result in the removal of any remaining C-terminal lysines.

“Linker” (“L”) refers to a substance (e.g., molecule) that binds the antibody to one or more drugs. The Linker can be a cleaveable linker or it can be a non-cleaveable linker. The linker is preferably non-cleavable. A non-cleavable linker keeps the drug attached to the antibody. Alternatively, for purposes of the present invention, the linker may e.g., couple, conjugate, join, connect, tether etc. the antibody to one or more drugs. In other embodiments, the binding of the linker to the antibody and drug is by means of a covalent bond.

“gp120” is defined as an envelope protein from HIV. This envelope protein is initially synthesized as a longer precursor protein of 845-870 amino acids in size, designated gp160. gp160 is cleaved by a cellular protease into gp120 and gp41. gp120 contains most of the external, surface-exposed, domains of the HIV envelope glycoprotein complex, and it is gp120 which binds both to cellular CD4 receptors and to cellular chemokine receptors (such as CCR5). See e.g., U.S. Patent Publication No. 20160009789.

“gp41” is defined as an HIV protein that contains a transmembrane domain and remains in a trimeric configuration; it interacts with gp120 in a non-covalent manner. The envelope protein of HIV-1 is initially synthesized as a longer precursor protein of 845-870 amino acids in size, designated gp160. gp160 forms a homotrimer and undergoes glycosylation within the Golgi apparatus. In vivo, it is then cleaved by a cellular protease into gp120 and gp41. The amino acid sequence of an example of gp41 is set forth in GENBANK® Accession No. CAD20975 (as available on Oct. 16, 2009) which is incorporated by reference herein (SEQ ID NO:1). It is understood that the sequence of gp41 can vary from that given in GENBANK® Accession No. CAD20975. gp41 contains a transmembrane domain and typically remains in a trimeric configuration; it interacts with gp120 in a non-covalent manner. See e.g., U.S. Patent Publication No. 20160009789 (gp120 vs gp41)

The term “gp160” refers to an envelope protein having a molecular weight of 160 kDa and contains various glycosylation sites. Gp160 acts as a precursor for both gp41 and gp120. For the purposes of the invention, gp160 is a representative envelope glycoprotein, and HXB2D is a non-limiting example of an envelope sequence. See e.g., https://www.hiv.1an1.gov/content/sequence/HIV/REVIEWS/HXB2.html, regarding HXB2D, the contents of which are incorporated by reference.

The term “envelope glycoprotein” or “glycoprotein” or “EnV” refers to a protein that contains oligosaccharide chains (glycans) covalently attached to polypeptide side-chains and that is exposed on the surface of the HIV envelope. For the purposes of the present invention, after administration of the antibody-drug conjugate to a subject, an HIV gp160 envelope glycoprotein is bound by the antibody-drug-conjugate. In some embodiments, the HIV gp160 envelope glycoprotein is bound to the antibody portion of the antibody-drug conjugate.

The term “broadly neutralizing antibody” (bNAb) is defined as an antibody which inhibits viral attachment and cell entry via binding to the HIV envelope glycoprotein (Env) (e.g., gp160), as a non-limiting example, by a 50% inhibition of infection in vitro, in more than 50%, 60%, 70%, 80%, 90%, 95%, 99% or greater, of a large panel of (greater than 100) HIV-1 envelope pseudotyped viruses and viral isolates. See e.g., US Published Patent Application No. 20120121597.

The term “drug” refers to an HIV therapeutic agent which encompasses e.g., a chemical compound or a larger molecule (e.g., a protein or a peptide) capable of inducing a desired therapeutic, treatment, or prophylactic effect with respect to HIV when properly administered to a subject or a cell. As an example, in one embodiment, the antibody-drug conjugate is a fused protein comprising one or more peptides fused to the C-terminal of the heavy and/or light chain and wherein the linker is 1 to 50 amino acids long.

For purposes of the present invention, one binding site that is targeted is the CD4 binding site. In various embodiments, the broadly neutralizing antibody Ab binds to the HIV envelope glycoprotein at the CD4 binding sight. As defined herein, CD4 is a Cluster of differentiation factor 4 polypeptide; a T-cell surface protein that mediates interaction with the MHC class II molecule. CD4 also serves as the primary receptor site for HIV on cells during HIV-I infection. CD4 is known to bind to gp120 from HIV. The known sequence of the CD4 precursor has a hydrophobic signal peptide, an extracellular region of approximately 370 amino acids, a highly hydrophobic stretch with significant identity to the membrane-spanning domain of the class II MHC beta chain, and a highly charged intracellular sequence of 40 resides (Maddon, Cell 42:93, 1985). The term “CD4” includes polypeptide molecules that are derived from CD4, including fragments of CD4, generated either by chemical (for example enzymatic) digestion or genetic engineering means. Such a fragment may be one or more entire CD4 protein domains. The extracellular domain of CD4 consists of four contiguous immunoglobulin-like regions (D1, D2, D3, and D4, see Sakihama et al., Proc. Natl. Acad. Sci. 92:6444, 1995; U.S. Pat. No. 6,117,655), and amino acids 1 to 183 have been shown to be involved in gp120 binding. For instance, a binding molecule or binding domain derived from CD4 would comprise a sufficient portion of the CD4 protein to mediate specific and functional interaction between the binding fragment and a native or viral binding site of CD4. One such binding fragment includes both the D1 and D2 extracellular domains of CD4 (DID2 is also a fragment of soluble CD4, or sCD4, which is comprised of D1 D2 D3 and D4), although smaller fragments may also provide specific and functional CD4-like binding. The gp120-binding site has been mapped to D1 of CD4. See e.g., US Published Patent Application No. 20120282264.

In another embodiment, the invention includes an antibody that binds HIV envelope glycoprotein at the gp120-gp41 interface. Such antibodies including, without limitation, an antibody selected from 8ANC195, 35022, and PGT151. An example of 8ANC195 is set forth in U.S. Publication No. 20150361160. An example of 35022 is set forth in U.S. Publication No. 20160022803. An example of PGT151 is set forth in U.S. Publication No. 20150152167.

In another embodiment, the invention includes an antibody that binds to the gp41 membrane-proximal external region (MPER) including, without limitation, 4E10, 10E8, 2F5 and Z13e1. An example of 4E10 is set forth in U.S. Publication No. 20160009789. An example of 10E8 is set forth in PCT Published Application No. WO2013070776. An example of 2F5 is set forth in U.S. Publication No. 20150158934. An example of Z13e1 is set forth in U.S. Publication No. 20120269821. A preferred antibody in this group is 10E8. Preferred antibodies employed in binding to the HIV envelope glycoprotein include without limitation VRC01, VRC07, VRC07-523, 3BNC117, NIH45-46, PGV04, b12, CH31, and CH103. In other embodiments, preferred antibodies include without limitation VRC01, VRC01-LS, VRC07, VRC07-LS, VRC07-523, 3BNC117, NIH45-46, PGV04, b12, CH31, CH103, N6, and N6-LS. A particularly preferred antibody is VRC01, an example of which is disclosed in Zhou et al., “Structural Basis for Broad and Potent Neutralization of HIV-1 by Antibody VRC01”, Science Express, 8 Jul. 2010, pp. 1-102, www.sciencemaq.org/cgi/content/full/science.1192819/DC1. More specifically, VRC01 may bind to the gp120. VRC01 is capable of neutralizing 90 percent of HIV strains/subtypes. Another example of such an antibody that binds to the gp120 is VRC01-LS, as disclosed in WO2012106578. Another example of such an antibody that binds to the gp120 is VRC07, as disclosed in WO2013086533.

An example of VRC07-523 is set forth in J. Virol, 88(21): pp. 12669-12682 (Nov. 2014). An example of 3BNC117 is set forth in U.S. Publication No. 20140212458. An example of NIH45-46 is set forth in U.S. Publication No. 20150274813. An example of PGV04 is set forth in U.S. Publication No. 20130251726. An example of b12 is set forth in U.S. Publication No. 20160009789. An example of CH31 is set forth in U.S. Publication No. 20130251726. An example of CH103 is set forth in U.S. Publication No. 20140212458.

In various embodiments, the broadly neutralizing antibody Ab is selected from the group consisting of 2G12, 2F5, 3BC176, 3BNC60, 3BNC117, 4E10, 8ANC131, 8ANC195, 10E8, 10-1074, 12A12, 35022, b12, B2530, CH01-04, CH103, CH31, HJ16, M66.6, N6, N6-LS, NIH45-46, PG9, PG16, PGDM1400, PGT121, PGT128, PGT135, PGT141-PGT145, PGT151, PGV04, VRC01, VRC01-LS, VRC07, VRC07-523, VRC07-LS, and Z13.

In view of the above, particularly preferred antibodies are VRC01, VRC01-LS, N6, N6-LS, VRC07 and VRC07-523. In addition to the above, an example of a disclosure of VRC01 is set forth in U.S. Pat. No. 8,637,036. An example of a disclosure of VRC01-LS is set forth in WO 2012/106578. Examples of disclosures of N6 and N6-LS are set forth in WO 2016/196975. Examples of disclosures of VRC07 and VRC07-523 are set forth in U.S. Pat. No. 8,637,036, US Patent Publication No. 2014/0322163 A1, WO 2016/196975 and WO2017/79479

In one embodiment, the broadly neutralizing antibody Ab binds to the HIV envelope glycoprotein selected from the group consisting of gp160, gp120 and gp41.

In one embodiment, the broadly neutralizing antibody Ab binds to the HIV envelope glycoprotein gp120.

In one embodiment, the broadly neutralizing antibody Ab binds to the HIV envelope glycoprotein gp41.

In an aspect of the invention the broadly neutralizing antibody comprises any one, two, there, four, five or all of the following CDRs: CDRH1 (SEQ ID NO:3), CDRH2 (SEQ ID NO:4), CDRH3 (SEQ ID NO:5), CDRL1 (SEQ ID NO:6), CDRL2 (SEQ ID NO:7) and CDRL3 (SEQ ID NO:8). In an embodiment of the invention the broadly neutralizing antibody comprises a heavy chain variable region of SEQ ID NO:9 and/or a light chain variable region of SEQ ID NO:10. In an embodiment of the invention the broadly neutralizing antibody comprises a leucine residue at position 428 of the heavy chain and a serine residue at position 434 of the heavy chain. In an embodiment of the invention the broadly neutralizing antibody comprises a heavy chain having at least 85%, 86%, 87%, 88%, 89%, 90%, 91%, 92%, 93%, 94%, 95%, 96%, 97%, 98%, 99%, or 100% sequence identity to SEQ ID NO:11 and/or a light chain having at least 85%, 86%, 87%, 88%, 89%, 90%, 91%, 92%, 93%, 94%, 95%, 96%, 97%, 98%, 99%, or 100% sequence identity to SEQ ID NO:13. In an embodiment of the invention, the broadly neutralizing antibody comprises a heavy chain of SEQ ID NO:12.

In an embodiment, the broadly neutralizing antibody comprises a heavy chain having at least 90% sequence identity to SEQ ID NO:9 and a light chain having at least 90% sequence identity to SEQ ID NO:10.

In an embodiment, the broadly neutralizing antibody comprises a heavy chain of SEQ ID NO:11, optionally comprising a light chain of SEQ ID NO:13 In an aspect of the invention the broadly neutralizing antibody comprises any one, two, there, four, five or all of the following CDRs: CDRH1 (SEQ ID NO:14), CDRH2 (SEQ ID NO:15), CDRH3 (SEQ ID NO:16), CDRL1 (SEQ ID NO:17), CDRL2 (SEQ ID NO:18) and CDRL3 (SEQ ID NO:19). In an embodiment, the broadly neutralizing antibody comprises a heavy chain having at least 90% sequence identity to SEQ ID NO:20 and a light chain having at least 90% sequence identity to SEQ ID NO:21. In an embodiment of the invention, the broadly neutralizing antibody comprises a heavy chain variable region of SEQ ID NO:20 and a light chain variable region of SEQ ID NO:21. In an embodiment of the invention, the broadly neutralizing antibody comprises a leucine residue at position 428 of the heavy chain and a serine residue at position 434 of the heavy chain. In an embodiment of the invention, the broadly neutralizing antibody comprises a heavy chain having at least 85%, 86%, 87%, 88%, 89%, 90%, 91%, 92%, 93%, 94%, 95%, 96%, 97%, 98%, 99%, or 100% sequence identity to SEQ ID NO:22 and a light chain having at least 85%, 86%, 87%, 88%, 89%, 90%, 91%, 92%, 93%, 94%, 95%, 96%, 97%, 98%, 99%, or 100% sequence identity to SEQ ID NO:23.

In accordance with the invention, a linker molecule is covalently bonded to the broadly neutralizing antibody. Examples of such linkers are known in the art and preferably include, for example, cleavable and non-cleavable linkers. Examples of non-cleavable linkers may include linkers that contain polyethylene glycol chains or polyethylene chains that are not acid or base sensitive (such as hydrazone containing linkers), are not sensitive to reducing or oxidizing agents (such as those containing disulfide linkages), and are not sensitive to enzymes that may be found in cells or in the circulatory system. See e.g., U.S. Pat. No. 8,470,980 and U.S. Patent Application 20090202536. Examples of particularly preferred linkers include, without limitation, those selected from the following structures below. In these embodiments, the linkers are illustrated in the context of various antibody-drug conjugates in accordance with the invention. The chemical moiety indicate as “bNAb” stands for the broadly neutrazling antibody that each linker is bonded to and at that position of the linker. Likewise, the term “drug” stands for the HIV attachment inhibitor compound that each linker is bonded to and at that position of the linker.

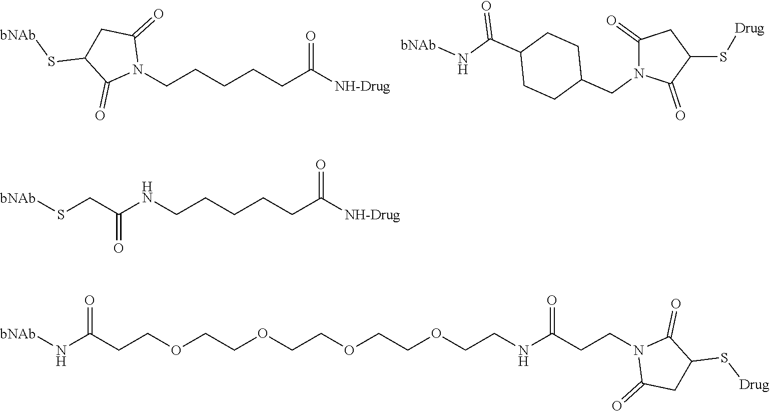

Other examples of linkers include, without limitation, those set forth below:

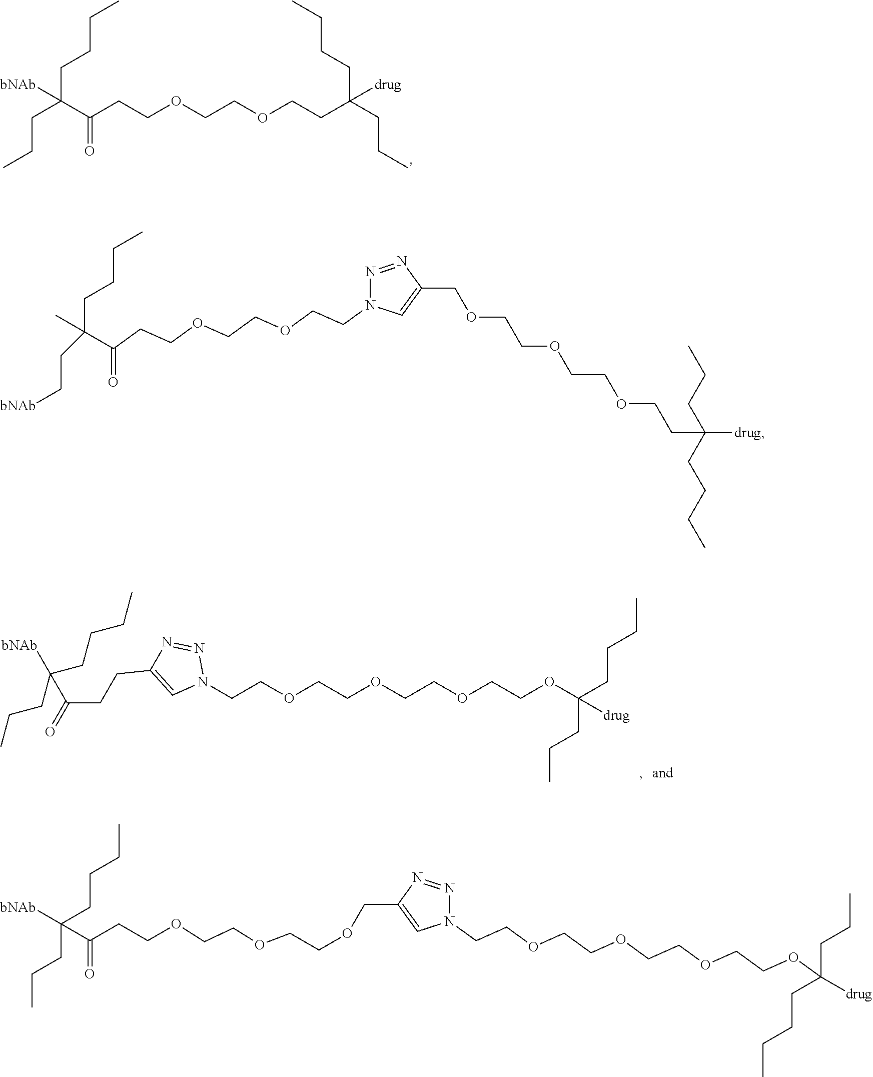

In the above embodiments, the bNAb and drug are each attached to the linker via various conjugations (e.g., cysteine and lysine). Others which are suitable may be used.

Examples of particularly suitable linkers and methods of attachment to antibody-drug conjugates are disclosed in Perez et al., Drug Discovery Today, Vol. 19, No. 7, (2014), pp. 869-881. As set forth therein, in one non-limiting example of a chemical conjugation, a reactive moiety pendant to the drug-linker may be covalently joined to the antibody via an amino acid residue side chain, commonly the e-amine of lysine. As demonstrated with Mylotarg1, direct conjugation of lysine residues on gemtuzumab can be achieved using an N-hydroxysuccini-mide (NHS) ester appended to the drug-linker to form stable amide bonds (see e.g., Bros, P. F., et al., Approval summary: gemtuzumab ozogamicin in relapsed acute myeloid leukemia, Clin. Cancer Res., 17, pp. 1490-1496 (2001). A two-step process can also be used in which surface lysines on the antibody are first modified to introduce a reactive group, such as a maleimide, and then conjugated to the drug-linker containing an appropriate reactive handle (e.g. a thiol) (see e.g., Junutula, J. R. et al., Site-specific conjugation of a cytotoxic drug to an antibody improves the therapeutic index, Nat. Biotechnol., 26: pp. 925-932 (2008). Various established site-specific conjugation methods known in the art can be used for making the ADCs. Such as, e.g., thiomab drug conjugation, antibody drug conjugates via transglutaminase, unnatural amino acids for antibody drug conjugates, SmarTag [see e.g., Christopher R Behrens & Bin Liu, Methods for site-specific drug conjugation to antibodies, mAbs, Vol 6, No. 1, pp. 46-53 (2014)]

In accordance with the invention, the antibody-drug conjugate includes one or more drugs covalently bonded to said linker molecule, said one or more drugs capable of binding to said HIV envelope glycoprotein. As one non-limiting example, the one or more drugs are selected from attachment inhibitors. This also emcompasses embodiments having a first drug covalently bonded to a first linker molecule covalently bonded to the bNAb, and a second drug covalently bonded to a second linker molecule covalently bonded to the bNAb. The term “attachment inhibitor” as used herein refers to drugs or agents (e.g., antiretrovirals) used for the treatment of HIV infection by interfering with the binding, fusion and entry of an HIV virion to a human cell. Examples of attachment inhibitors include, without limitation, gp120 attachment inhibitors and gp160 attachment inhibitors. Examples of attachment inhibitors include, without limitation, gp120 attachment inhibitors, gp160 attachment inhibitors, and gp41 attachment inhibitors. Not intending to be bound by theory, in one embodiment, attachment inhibitors target gp160 envelope protein (gp120+gp41). Examples of attachment inhibitors are azaindoleoxoacetyl pirerazine derivatives, and a particularly preferred attachment inhibitor is of the formula:

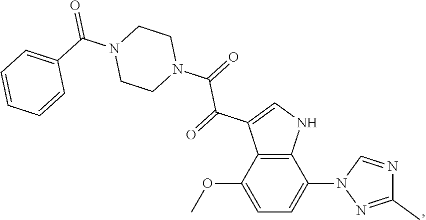 [SMILES: COc1ccc(-n2cnc(C)n2)c2[nH]cc(C(=O)C(=O)N3CCN(C(=O)c4ccccc4)CC3)c12]

as set forth in U.S. Pat. Nos. 7,501,420; 7,354,924, and 7,662,823.

Other examples of drugs include, without limitation, peptides (e.g., as described in U.S. Pat. Nos. 6,133,418 and 6,475,491. For example the drug may be a peptide that binds to CD4. A preferred example of such a drug is set forth as SEQ ID NO:2 below:

Ac-Tyr-Thr-Ser-Leu-Ile-His-Ser-Leu-Ile-Glu-Glu-Ser-GIn-Asn-GIn-GIn-Glu-Lys-Asn-Glu-Gln-Glu-Leu-Leu-Glu-Leu-Asp-Lys-Trp-Ala-Ser-Leu-Trp-Asn-Trp-Phe-NH₂

SEQ ID NO: 2 is known as T-20 marketed by Roche under the name FUZEON®.

Other examples of suitable compounds include, e.g., that set forth below:

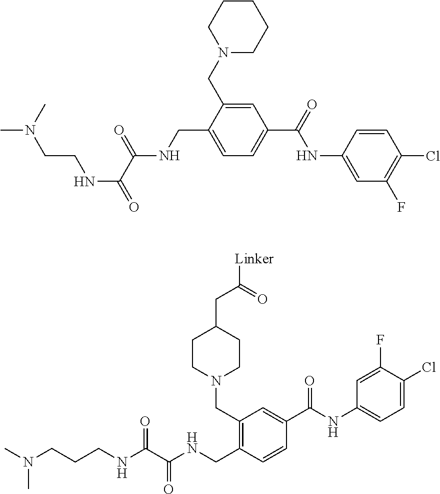

which are gp160 attachment inhibitors

The invention also provides compounds of Formula A that may be used as drugs in the antibody-drug-conjugates disclosed herein:

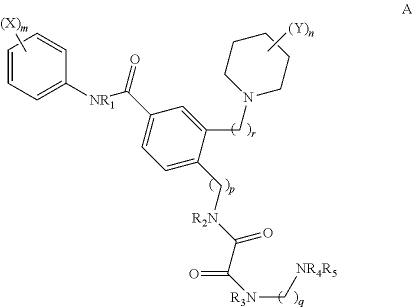

wherein:

X and Y are independently selected from the group consisting of of H, (C₁-C₆)alkyl, (C₁-C₆)alkoxy, halo, oxo, haloalkyl, bihaloalkyl, trihaloalkyl, haloalkoxy, bihaloalkoxy, trihaloalkoxy, hydroxyl, amino, amide and (C₁-C₆)alkyl-(C═O)—(C1-C6);

R₁, R₂, R₃, R₄ and R₅ are each independently selected from H or (C₁-C₆)alkyl;

m ranges from 0 to 5; more preferably 1 to 4;

n ranges from 0 to 5; more preferably 1 to 4;

r ranges from 0 to 6, more preferably 1 to 6, most preferably 1 to 4;

p ranges from 0 to 6, more preferably 1 to 6, most preferably 1 to 4; and

q ranges from 0 to 6, more preferably 1 to 6, most preferably 1 to 4;

wherein the compound of formula A can be attached to a linker via R₄ or R₅; or Y.

In one embodiment with respect to the compound of formula A:

X is selected from Cl and F; and m is 2;

### Y is H;

R₁, R₂, R₃, R₄ and R₅ are each independently H;
r ranges from 1 to 4; most preferably is 1;
p ranges from 1 to 4; most preferably is 1; and
q ranges from 1 to 4; most preferably is 2.

A preferred compound of formula A is:

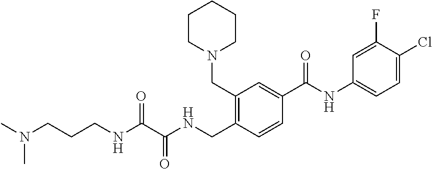 [SMILES: CN(C)CCCNC(=O)C(=O)NCc1ccc(C(=O)Nc2ccc(Cl)c(F)c2)cc1CN1CCCCC1]

Such compounds may be made according to the following synthesis:

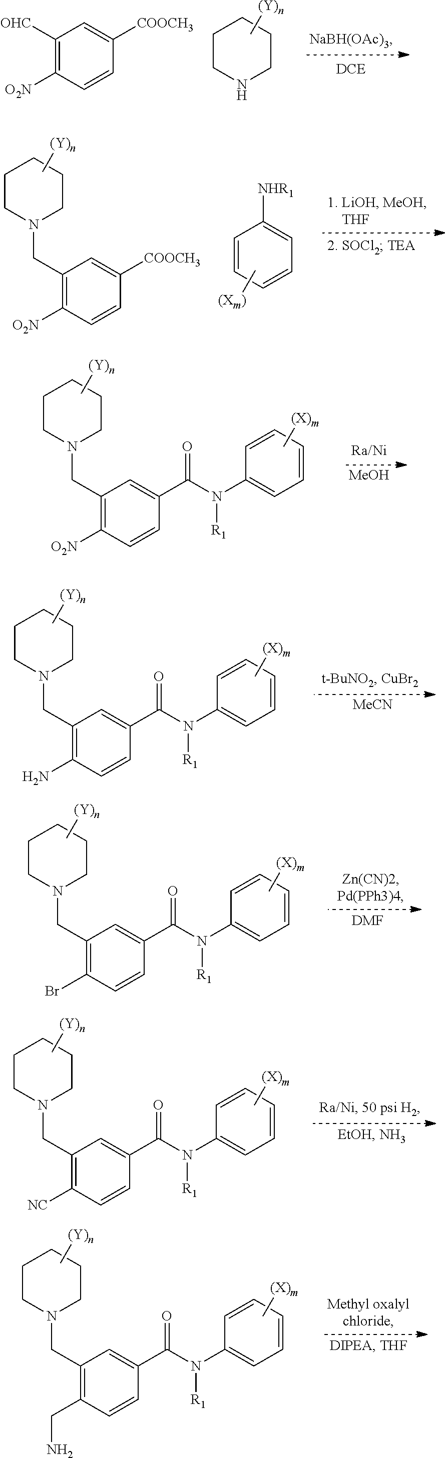

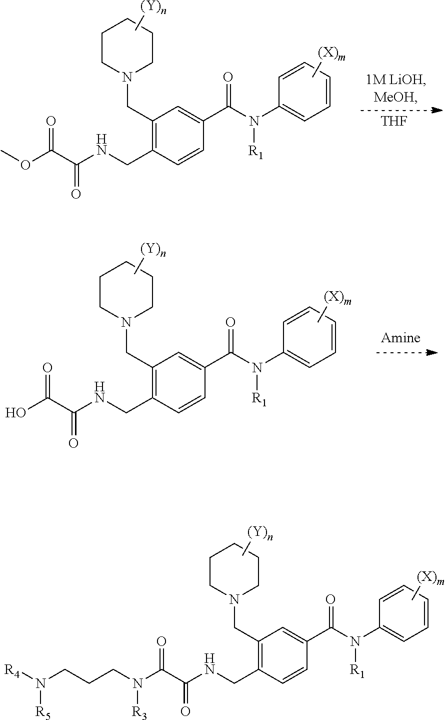

Wherein X, Y, m, n, R₁ and R₄ are defined herein above.

Examples of drug-linker pairs include, without limitation, that may be used in conjunction with an antibody in accordance with the invention are as follows:

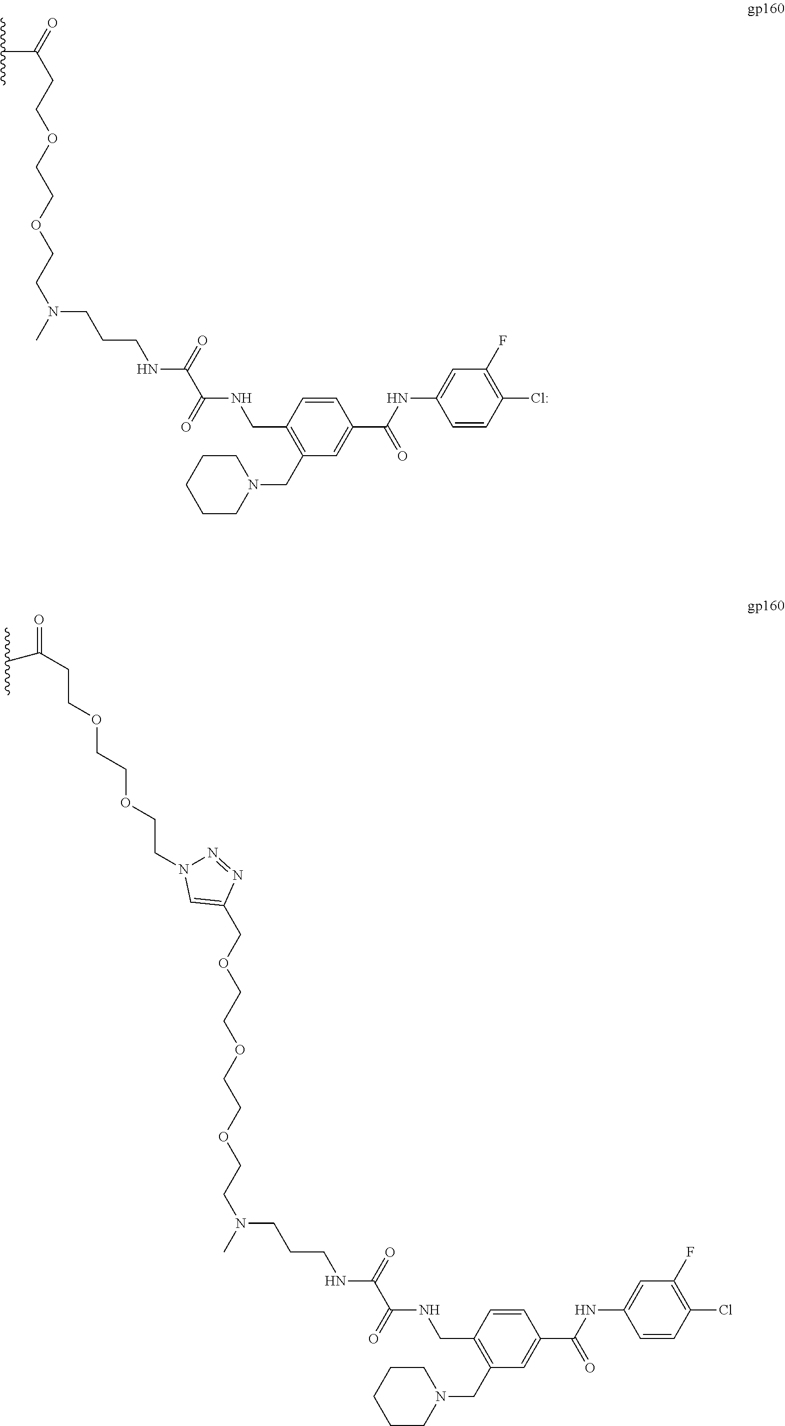

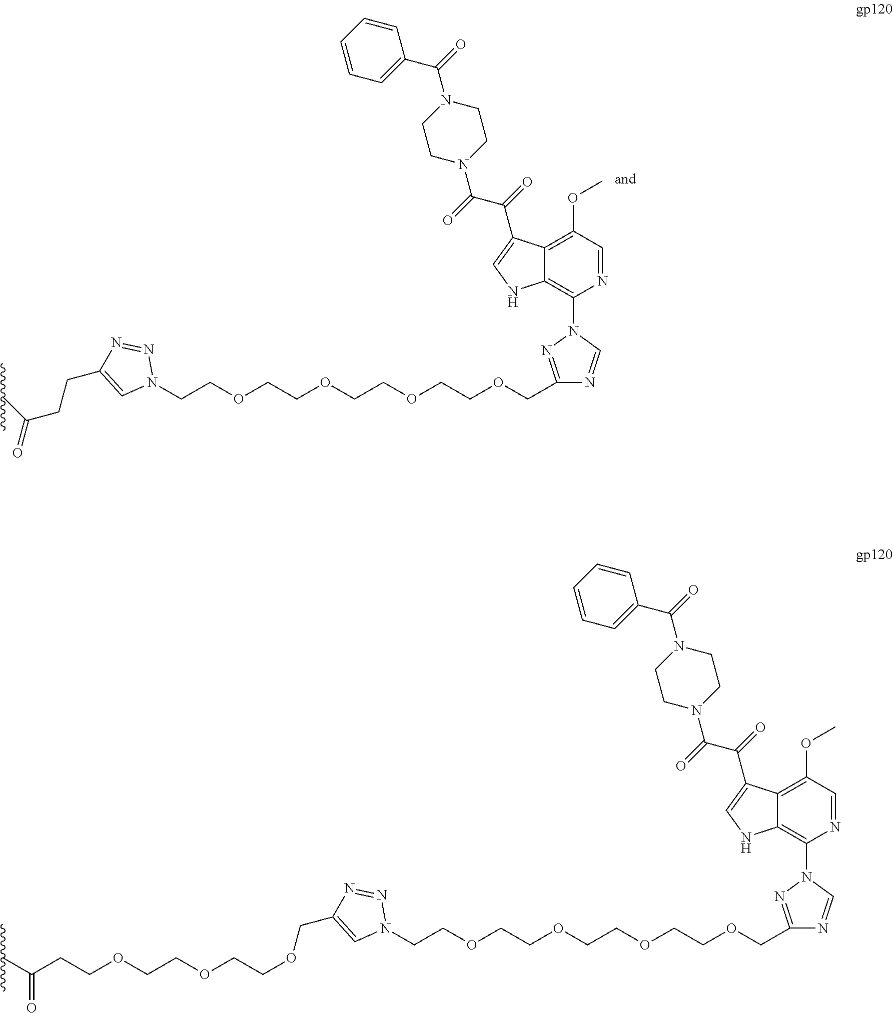 [SMILES: *C(=O)CCc1cn(CCOCCOCCOCCOCc2ncn(-c3ncc(OC)c4c(C(=O)C(=O)N5CCN(C(=O)c6ccccc6)CC5)c[nH]c34)n2)nn1 ; *C(=O)CCOCCOCCOCc1cn(CCOCCOCCOCCOCc2ncn(-c3ncc(OC)c4c(C(=O)C(=O)N5CCN(C(=O)c6ccccc6)CC5)c[nH]c34)n2)nn1]

wherein

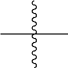

represents attachment to the bNAb.
Specific embodiments of antibody-drug-conjugates are as follows:

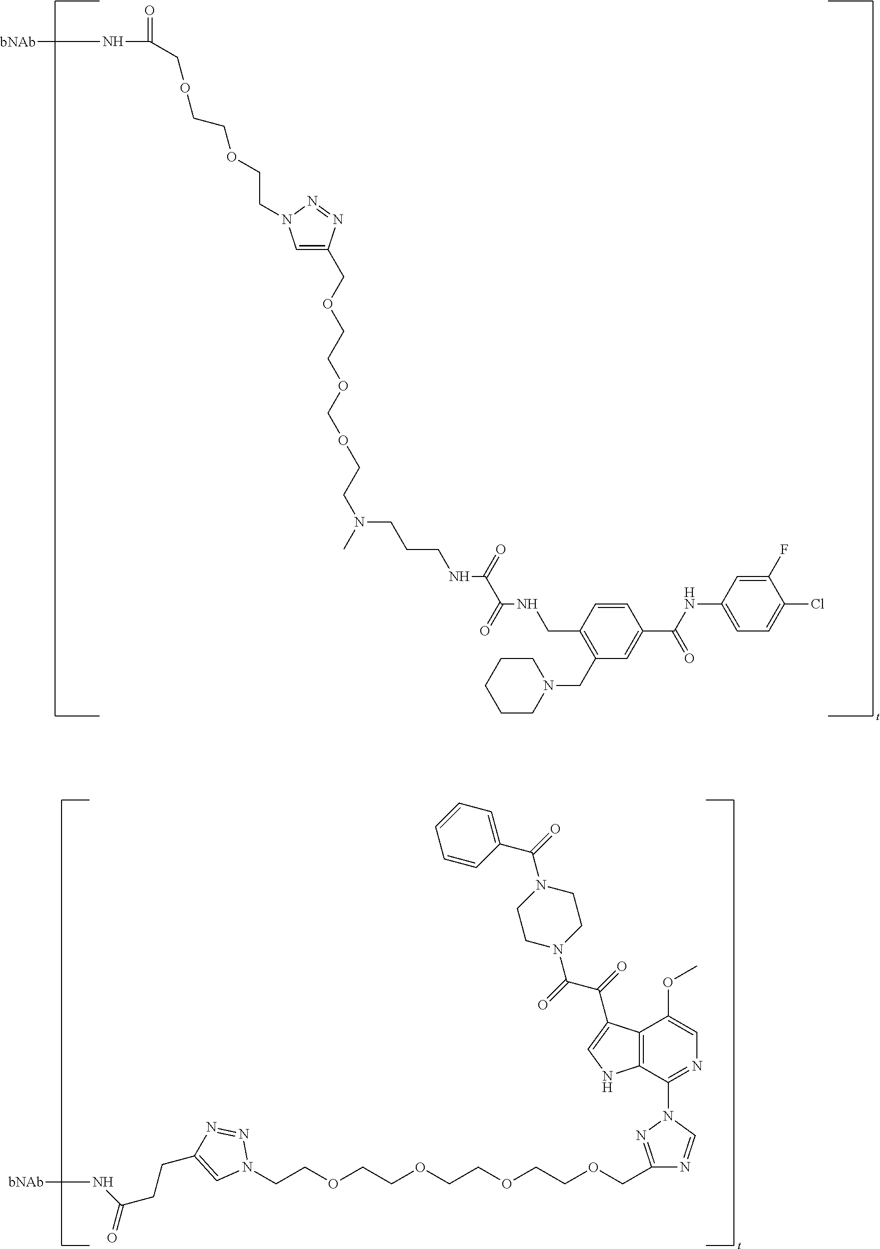

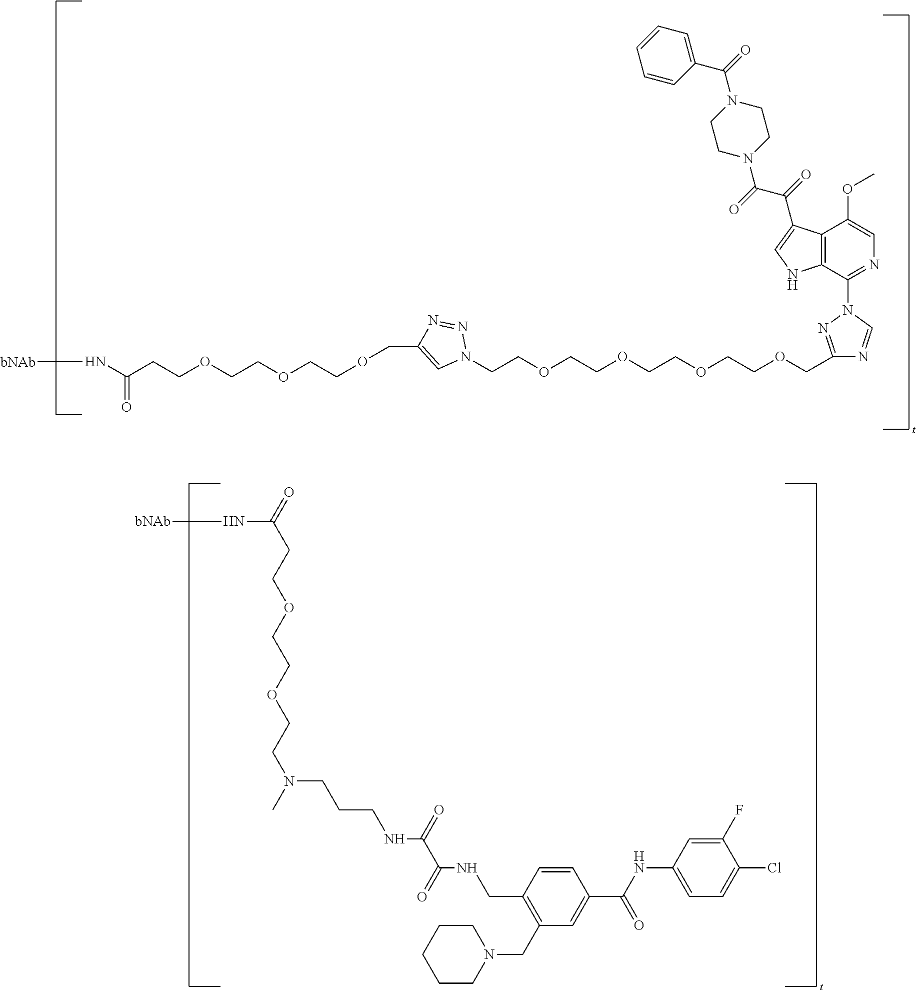

Wherein t ranges from 1 to 12.

Examples of current compounds and agents for HIV treatment include various other entry and fusion inhibitors, such as AMD070, BMS-488043, Fozivudine tidoxil, GSK-873,140 (aplaviroc), PRO 140, PRO 542, Peptide T, SCH-D (vicriviroc), TNX-355, and UK-427,857 (maraviroc); integrase inhibitors, such as GS 9137, MK-0518, as set forth in U.S. Pat. No. 9,259,433,

In one non-limiting aspect, the present invention encompasses antibody-drug conjugates in which a linker bonds an antibody to an agent through an attachment at a particular amino acid within the antibody or antigen-binding molecule. See e.g., U.S. Pat. No. 9,302,015. Exemplary amino acid attachments that can be used in the context of this aspect of the invention include, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313).

In an embodiment wherein the drug in the antibody-drug conjugate is a peptide or a polypeptide, e.g. a DAB, the linker may be an amino acid linker which links the drug peptide or drug polypeptide to the antibody at one or both of the antibody heavy chains or one or both of the antibody light chains resulting in a fusion protein. In an embodiment, the drug peptide or drug polypeptide is fused to the C terminal of one or both of the heavy chains of the antibody. In an embodiment the amino acid linker is between 0 and 150 amino acids long, more specifically, as an example in another embodiment, between 0 and 50 amino acids.

In another aspect, as defined herein, the invention encompasses antibody-drug conjugates wherein one or more drugs are attached in two or more discrete locations to the antibody. Such an aspect may encompass, without limitation, any of the antibodies, linkers and drugs defined herein.

Specific examples of such antibody drug conjugates are, without limitation:

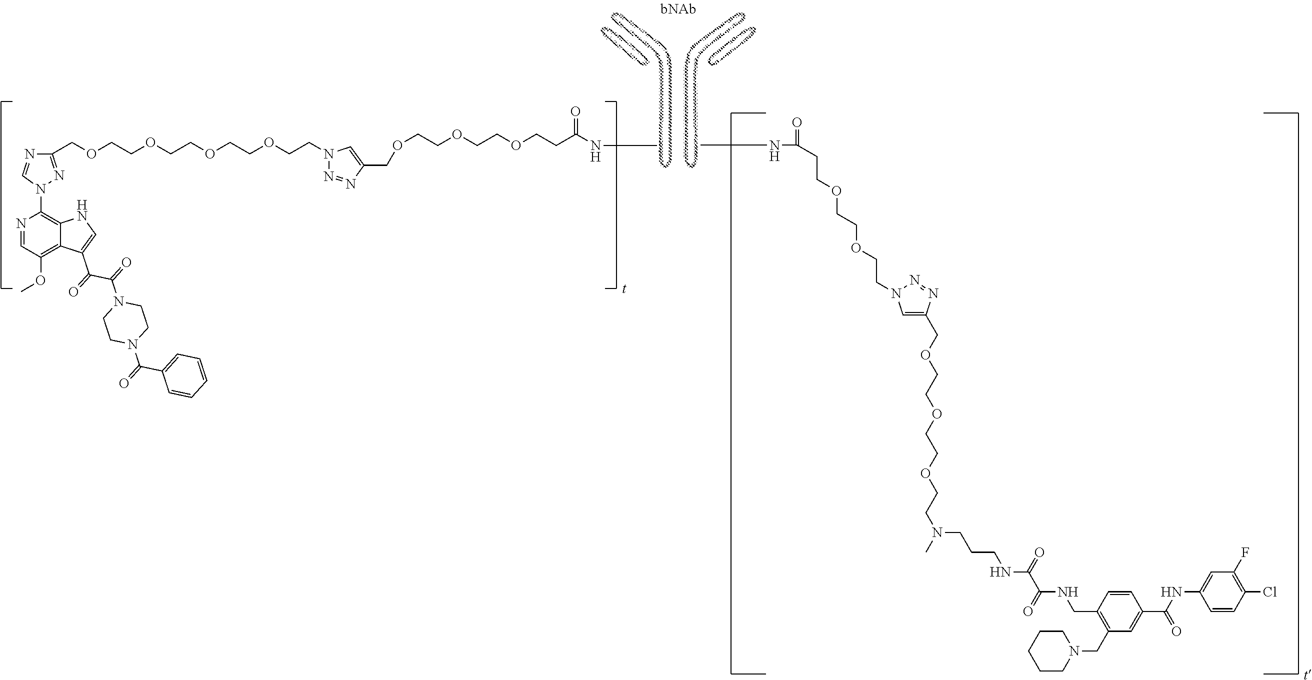

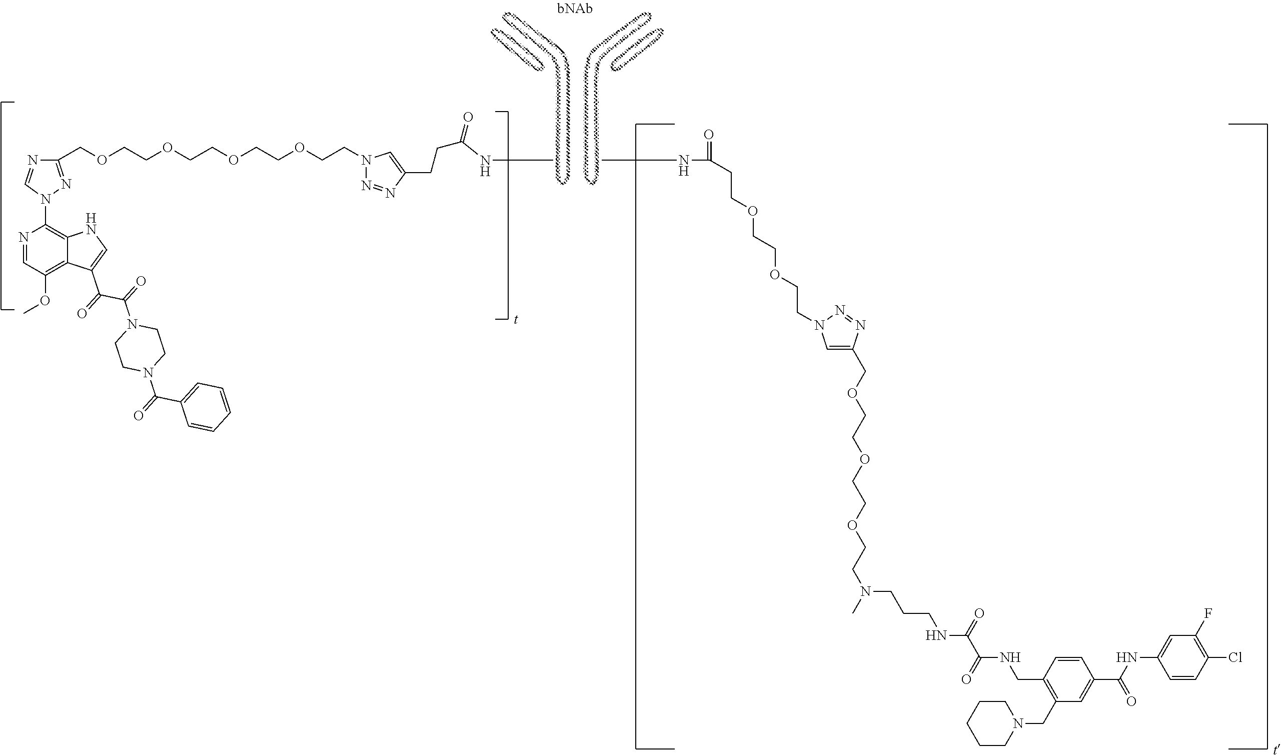

Wherein t and t′ each independently range from 1 to 12.

“Cure” or “Curing” a disease in a patient is used to denote the eradication, stoppage, halt or end of the human immunodeficiency virus or symptoms, or the progression of the symptoms or virus, for a defined period. As an example, in one embodiment, “cure” or “curing” refers to a therapeutic administration or a combination of administrations that alone or in combination with one or more agents induces and maintains sustained viral control (undetectable levels of plasma viremia by, e.g., a polymerase chain reaction (PCR) test, a bDNA (branched chain DNA) test or a NASBA (nucleic acid sequence based amplification) test) of human immunodeficiency virus after a minimum of, by way of example, one or two years without any other therapeutic intervention. The above PCR, bDNA and NASBA tests are carried out using techniques known and familiar to one skilled in the art. As an example, the eradication, stoppage, halt or end of the human immunodeficiency virus or symptoms, or the progression of the symptoms or virus, may be sustained for a minimum of two years.

Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

In accordance with one embodiment of the present invention, there is provided a pharmaceutical composition comprising an antibody-drug conjugate as set forth herein and a pharmaceutically acceptable excipient.

In accordance with one embodiment of the present invention, there is provided a method of curing an HIV infection in a subject comprising administering to the subject an antibody-drug conjugate as described herein.

In accordance with one embodiment of the present invention, there is provided a method of curing an HIV infection in a subject comprising administering to the subject a pharmaceutical composition as described herein.

In accordance with one embodiment of the present invention, there is provided a method of treating an HIV infection in a subject comprising administering to the subject an antibody-drug conjugate as described herein.

In accordance with one embodiment of the present invention, there is provided a method of treating an HIV infection in a subject comprising administering to the subject a pharmaceutical composition as described herein.

In accordance with one embodiment of the present invention, there is provided a method of preventing an HIV infection in a subject at risk for developing an HIV infection, comprising administering to the subject an antibody-drug conjugate as described herein.

In accordance with one embodiment of the present invention, there is provided a method of preventing an HIV infection in a subject at risk for developing an HIV infection, comprising administering to the subject a pharmaceutical composition as described herein.

In another embodiment of the present invention, there is provided an antibody-drug conjugate, as described herein, for use as a medicament.

In another embodiment of the present invention, there is provided an antibody-drug-conjugate, as described herein, for use in curing an HIV infection.

In another embodiment of the present invention, there is provided an antibody-drug-conjugate, as described herein, for use in treating an HIV infection.

In another embodiment of the present invention there is provided an antibody-drug-conjugate, as described herein, for use in preventing an HIV infection.

In another embodiment of the invention, there is provided an antibody-drug conjugate, wherein the same is used in the manufacture of a medicament for use in the treatment of an HIV infection in a human.

In another embodiment of the invention, there is provided an antibody-drug conjugate, wherein the same is used in the manufacture of a medicament for use in the prevention of an HIV infection in a human.

In another embodiment of the invention, there is provided an antibody-drug conjugate wherein the same or salt of the compound is used in the manufacture of a medicament for use in the cure of an HIV infection in a human.

In one embodiment, the pharmaceutical formulation containing antibody-drug conjugate is a formulation adapted for parenteral administration. In another embodiment, the formulation is a long-acting parenteral formulation.

The antibody-drug conjugates of the invention may be employed alone or in combination with other therapeutic agents. Therefore, in other embodiments, the methods of treating and/or preventing an HIV infection in a subject may in addition to administration of an antibody-drug conjugate further comprise administration of one or more additional pharmaceutical agents active against HIV.

In such embodiments, the one or more additional agents active against HIV is/are selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, vicriviroc (Sch-C), Sch-D, TAK779, maraviroc, TAK449, didanosine, tenofovir, lopinavir, and darunavir.

As such, the antibody-drug conjugates of the present invention and any other pharmaceutically active agent(s) may be administered together or separately and, when administered separately, administration may occur simultaneously or sequentially, in any order. The amounts of the antibody-drug conjugates of the present invention and the other pharmaceutically active agent(s) and the relative timings of administration will be selected in order to achieve the desired combined therapeutic effect. The administration in combination of antibody-drug conjugates with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the antibody-drug conjugates and the other pharmaceutically active agent(s) and the relative timings of administration will be selected in order to achieve the desired combined therapeutic effect.

In addition, the antibody-drug conjugates may be used in combination with one or more other agents that may be useful in the prevention, treatment or cure of HIV. Examples of such agents include:

Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, TDF, TAF
and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Integrase inhibitors such as raltegravir, elvitegravir, bictegravir, dolutegravir, cabotegravir and similar agents;
Maturation inhibitors such as PA-344 and PA-457, and similar agents; and GSK2838232.
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.
Further examples where the antibody-drug conjugates of the present invention may be used in combination with one or more agents useful in the prevention or treatment of HIV are found in Table 2.

TABLE 2

| | | | |
|---|---|---|---|
| | Brand | | |
| FDA Approval | Name | Generic Name | Manufacturer |

Nucleoside Reverse
Transcriptase Inhibitors
(NRTIs)

| | | | |
|---|---|---|---|
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, ddI | Squibb |
| 1992 | Hivid | zalcitabine, | Roche |
| | | dideoxycytidine, | Pharmaceuticals |
| | | ddC | |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + | GlaxoSmithKline |
| | | zidovudine | |
| 1998 | Ziagen | abacavir sulfate, | GlaxoSmithKline |
| | | ABC | |
| 2000 | Trizivir | abacavir + | GlaxoSmithKline |
| | | lamivudine + | |
| | | zidovudine | |
| 2000 | Videx EC | enteric coated | Bristol-Myers |
| | | didanosine, ddI EC | Squibb |
| 2001 | Viread | tenofovir disoproxil | Gilead Sciences |
| | | fumarate, TDF | |
| 2003 | Emtriva | emtricitabine, FTC | Gilead Sciences |
| 2004 | Epzicom | abacavir + | GlaxoSmithKline |
| | | lamivudine | |
| 2004 | Truvada | emtricitabine + | Gilead Sciences |
| | | tenofovir disoproxil | |
| | | fumarate | |

Non-Nucleosides
Reverse Transcriptase
Inhibitors (NNRTIs)

| | | | |
|---|---|---|---|
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |

Protease Inhibitors (PIs)

| | | | |
|---|---|---|---|
| 1995 | Invirase | saquinavir | Roche |
| | | mesylate, SQV | Pharmaceuticals |
| 1996 | Norvir | ritonavir, RTV | Abbott Laboratories |
| 1996 | Crixivan | indinavir, IDV | Merck |
| 1997 | Viracept | nelfinavir mesylate, | Pfizer |
| | | NFV | |
| 1997 | Fortovase | saquinavir (no | Roche |
| | | longer marketed) | Pharmaceuticals |
| 1999 | Agenerase | amprenavir, APV | GlaxoSmithKline |
| 2000 | Kaletra | lopinavir + ritonavir, | Abbott Laboratories |
| | | LPV/RTV | |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS-APV | |
| 2005 | Aptivus | tripranavir, TPV | Boehringer |
| | | | Ingelheim |
| 2006 | Prezista | Darunavir | Tibotec |
| | | | Therapeutics |

Fusion Inhibitors

| | | | |
|---|---|---|---|
| 2003 | Fuzeon | Enfuvirtide, T-20 | Roche |
| | | | Pharmaceuticals & |
| | | | Trimeris |

Entry Inhibitors

| | | | |
|---|---|---|---|
| 2007 | Selzentry | Maraviroc | Pfizer |

Integrase Inhibitors

| | | | |
|---|---|---|---|
| 2007 | Isentress | Raltegravir | Merck |
| 2013 | Tivicay | Dolutegravir | ViiV Healthcare |
| — | — | Cabotegravir | |

The scope of combinations of antibody-drug conjugates of this invention with HIV agents is not limited to those mentioned above, but includes in principle any combination with any pharmaceutical composition useful for the cure, treatment and/or prevention of HIV. As noted, in such combinations the antibody-drug conjugates of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350, and SPI-452. Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott park, Illinois, as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

GS-9350 is a compound being developed by Gilead Sciences of Foster City Calif. as a pharmacological enhancer.

SPI-452 is a compound being developed by Sequoia Pharmaceuticals of Gaithersburg, Md., as a pharmacological enhancer.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, an antibody-drug conjugate.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, an antibody-drug conjugate, wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, an antibody-drug conjugate, further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, an antibody-drug conjugate, further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors;

Protease inhibitors; Entry, attachment and fusion inhibitors; Integrase inhibitors; Maturation inhibitors; CXCR4 inhibitors; and CCR5 inhibitors.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, an antibody-drug conjugate.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, an antibody-drug conjugate, wherein said virus is an HIV virus. In some embodiments, the HIV virus is the HIV-1 virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, an antibody-drug conjugate, further comprising administration of a therapeutically effective amount of one or more agents active against an HIV virus.

In another embodiment of the invention, there is provided a method for curing a viral infection in a mammal mediated at least in part by a virus in the retrovirus family of viruses which method comprises administering to a mammal, that has been diagnosed with said viral infection or is at risk of developing said viral infection, an antibody-drug conjugate.

In another embodiment of the invention, there is provided a method for curing a viral infection in a mammal mediated at least in part by a virus in the retrovirus family of viruses which method comprises administering to a mammal, that has been diagnosed with said viral infection or is at risk of developing said viral infection, an antibody-drug conjugate, wherein said virus is an HIV virus. In some embodiments, the HIV virus is the HIV-1 virus.

In another embodiment of the invention, there is provided a method for curing a viral infection in a mammal mediated at least in part by a virus in the retrovirus family of viruses which method comprises administering to a mammal, that has been diagnosed with said viral infection or is at risk of developing said viral infection, an antibody-drug conjugate, further comprising administration of a therapeutically effective amount of one or more agents active against an HIV virus.

In another embodiment of the invention, there is provided a method for curing a viral infection in a mammal mediated at least in part by a virus in the retrovirus family of viruses which method comprises administering to a mammal, that has been diagnosed with said viral infection or is at risk of developing said viral infection, an antibody-drug conjugate, further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In another embodiment, there is provided a pharmaceutical composition comprising a pharmaceutically acceptable diluent and a therapeutically effective amount of an antibody-drug conjugate.

As used herein, the term “pharmaceutically acceptable” refers to those antibody-drug conjugates, agents, compounds, materials, compositions, and dosage forms which are, within the scope of sound medical judgment, suitable for use in contact with the tissues of human beings and animals without excessive toxicity, irritation, or other problem or complication.

Administration of the drugs described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used. One example of an administration is an intravenous administration, in which instance a pharmaceutical formulation suitable for intravenous administration is employed. Another example of an administration is an intramuscular administration, in which instance a pharmaceutical formulation suitable for intramuscular administration is employed. Another example of an administration is an subcutaneuous administration, in which instance a pharmaceutical formulation suitable for subcutaneous administration is employed.

Pharmaceutical compositions or formulations include solid, semi-solid, liquid and aerosol dosage forms, such as, e.g., tablets, capsules, powders, liquids, suspensions, suppositories, aerosols or the like useful in any of the above administrations. The antibody-drug conjugates can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The antibody-drug conjugates described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like).

Generally, depending on the intended mode of administration, the pharmaceutical composition will contain about 0.005% to 95%; in certain embodiments, about 0.5% to 50% by weight of a ADC. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one antibody-drug conjugate and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of antibody-drug conjugate contained in such parenteral compositions is highly dependent on the specific nature thereof, as well as the activity of the chemical entities and the needs of the subject.

However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the antibody-drug conjugate described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the antibody-drug conjugates provided will be administered in a therapeutically effective amount by any of the accepted modes of administration for agents that serve similar utilities. The actual amount of the antibody-drug conjugate will depend upon numerous factors such as the severity of the disease to be treated, the age and relative health of the subject, the potency of the antibody-drug conjugate used the route and form of administration, and other factors. The antibody-drug conjugate can be administered more than once a day, such as once or twice a day.

Therapeutically effective amounts of the antibody-drug conjugate described herein may range from approximately 0.01 to 200 mg per kilogram body weight of the recipient per day; such as about 0.01-100 mg/kg/day, for example, from about 0.01 to 50 mg/kg/day. Thus, for administration to a 70 kg person, the dosage range may be about 1-1000 mg per day.

In general, the antibody-drug conjugates will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used.

Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the antibody-drug conjugate. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one antibody-drug conjugate described herein in combination with at least one pharmaceutically acceptable excipient. Acceptable excipients are non-toxic, aid administration, and do not adversely affect the therapeutic benefit of the at least one active agent described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse an antibody-drug conjugate described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the antibody-drug conjugate in a composition can vary within the full range employed by those skilled in the art. Typically, the composition will contain, on a weight percent (wt %) basis, from about 0.01-99.99 wt % of antibody-drug conjugate entity described herein based on the total composition, with the balance being one or more suitable pharmaceutical excipients. In certain embodiments, the antibody-drug conjugate described herein is present at a level of about 1-80 wt %.

Various broadly neutralizing antibodies (bnAbs) have been explored as ARVs by infusion into HIV infected individuals or relevant models with limited success. The term “ARVs” refers to “antiretrovirals” which are drugs for the treatment of infection by a retrovirus, namely HIV, to inhibit the reproduction of such a virus. Resistance to bnAbs is generated during treatment similar to that observed with small molecule ARVs. To this end, a bi-functional molecule comprised of a bnAb and a small molecule attachment inhibitor targeting gp160 in accordance with the invention is believed to be capable of increasing the breadth of gp160 diversity inhibited and improve durability by providing multiple anti-viral targets in one molecule analogous to HAART provided by multiple small molecules. The term “HAART” refers to “highly active anti-retroviral therapy” which is the combination of more than one (e.g., 2, 3 or 4) drugs for the treatment of HIV.

### Example 1

### Synthesis of gp160 Attachment Inhibitor

The following route was employed to make a drug used in an antibody-drug-conjugate in accordance with the invention (Scheme 1):

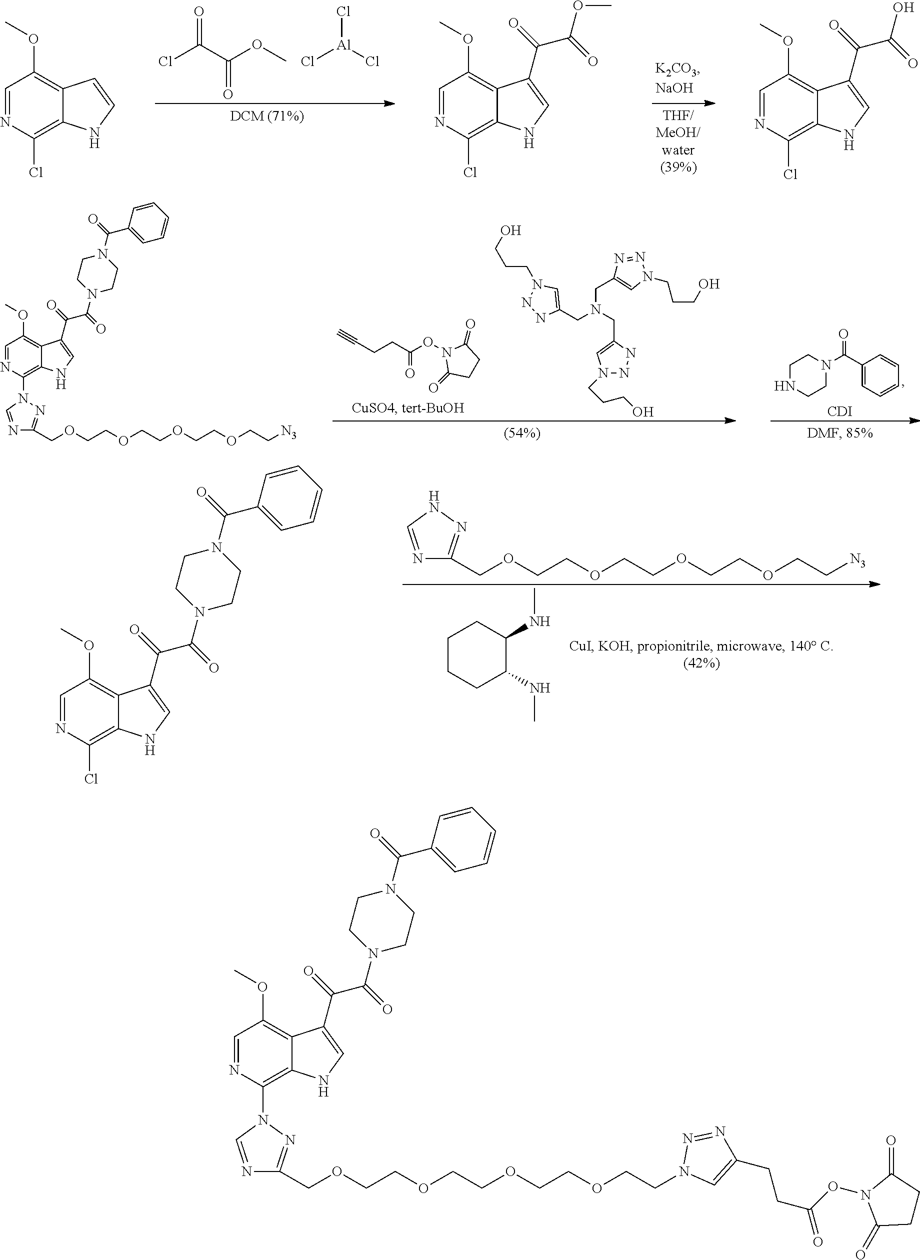

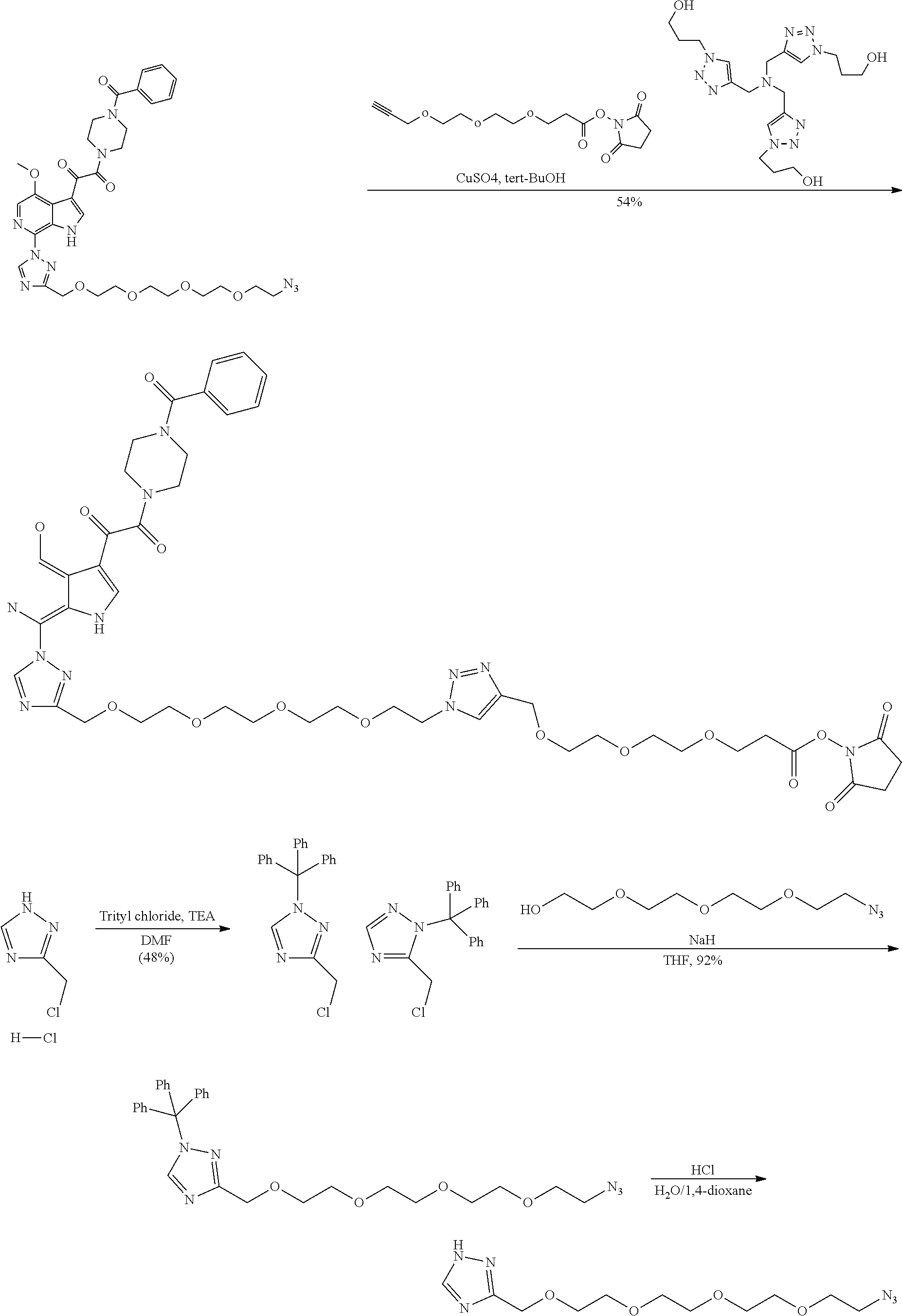

### Example 2

### Experimental Procedure

A conjugator A to VRC01 with a gp160 inhibitor and linker was made following the Scheme 1-4. In this example, a lysine conjugation was carried out with VRC01; therefore, a succinimidyl ester was incorporated into the conjugator. As a surrogate to attempt to validate the biological activity after all these modifications, compound B was also made.

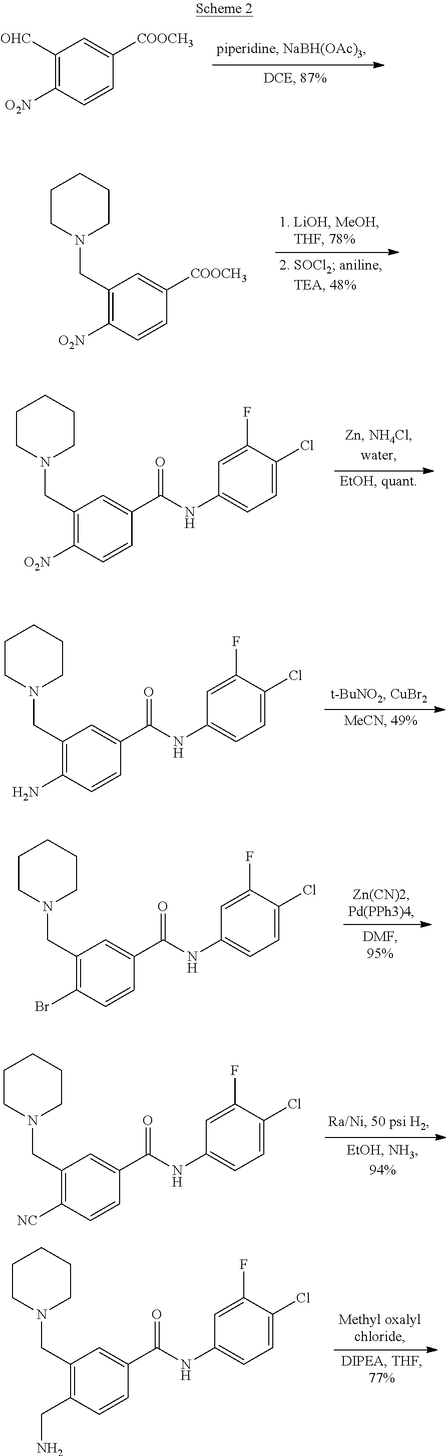

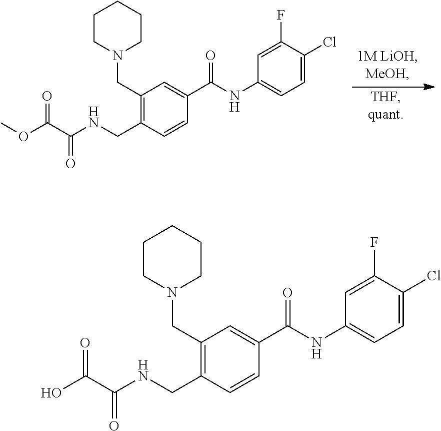 [SMILES: COC(=O)C(=O)NCc1ccc(C(=O)Nc2ccc(Cl)c(F)c2)cc1CN1CCCCC1 ; "THF,quant." ; O=C(O)C(=O)NCc1ccc(C(=O)Nc2ccc(Cl)c(F)c2)cc1CN1CCCCC1]

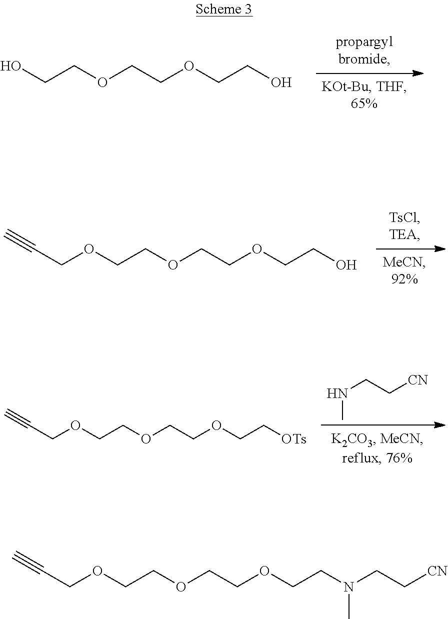

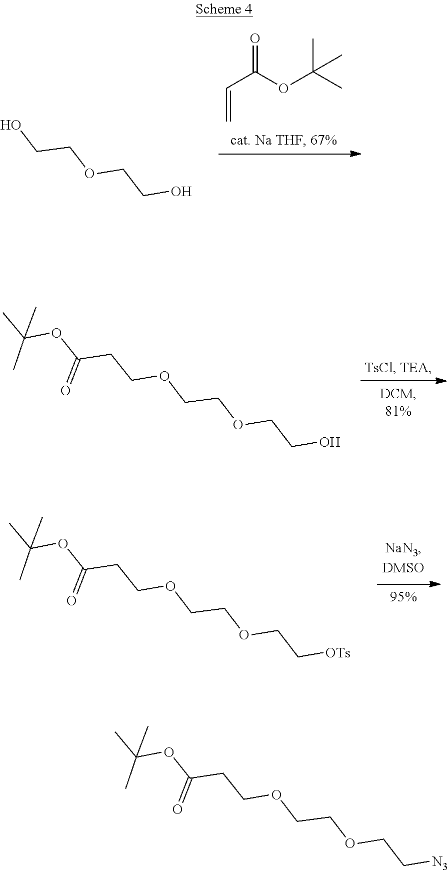

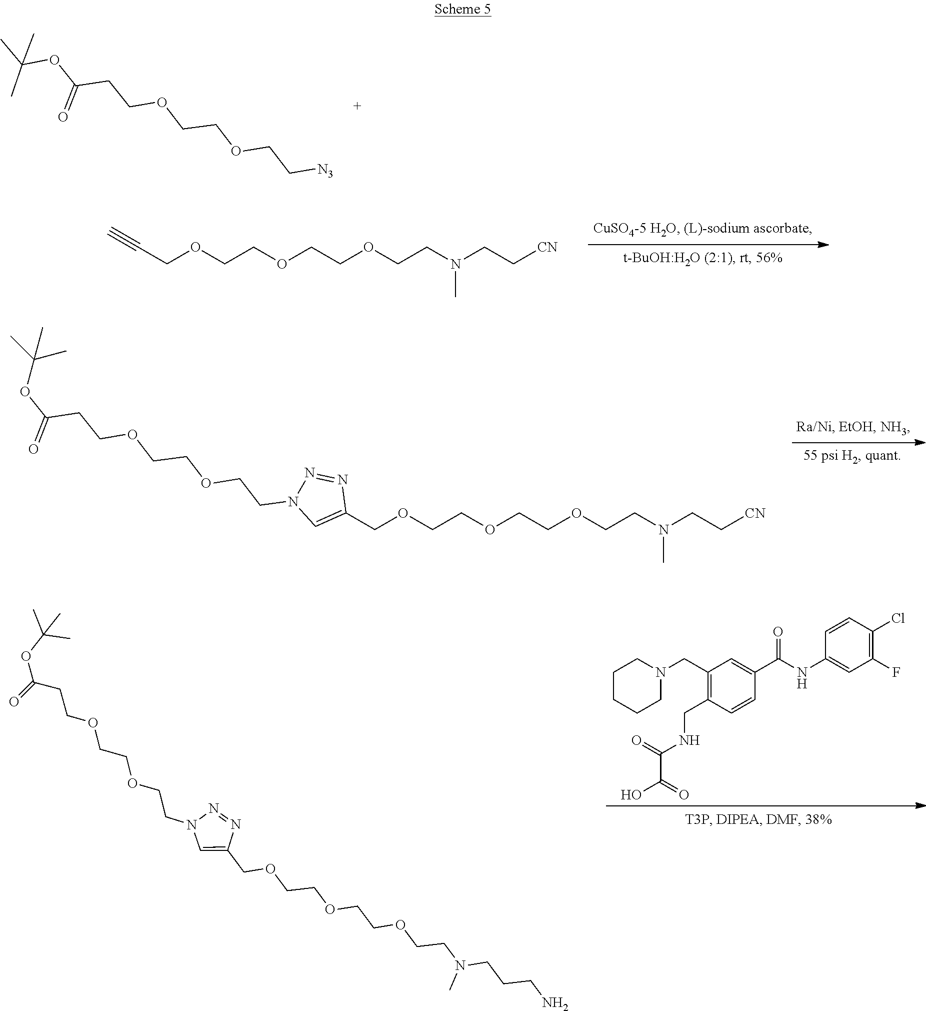 [SMILES: CC(C)(C)OC(=O)CCOCCOCCN=[N+]=[N-] ; C#CCOCCOCCOCCN(C)CCC#N ; CN(CCC#N)CCOCCOCCOCc1cn(CCOCCOCCC(=O)OC(C)(C)C)nn1 ; CN(CCCN)CCOCCOCCOCc1cn(CCOCCOCCC(=O)OC(C)(C)C)nn1 ; O=C(O)C(=O)NCc1ccc(C(=O)Nc2ccc(Cl)c(F)c2)cc1CN1CCCCC1]

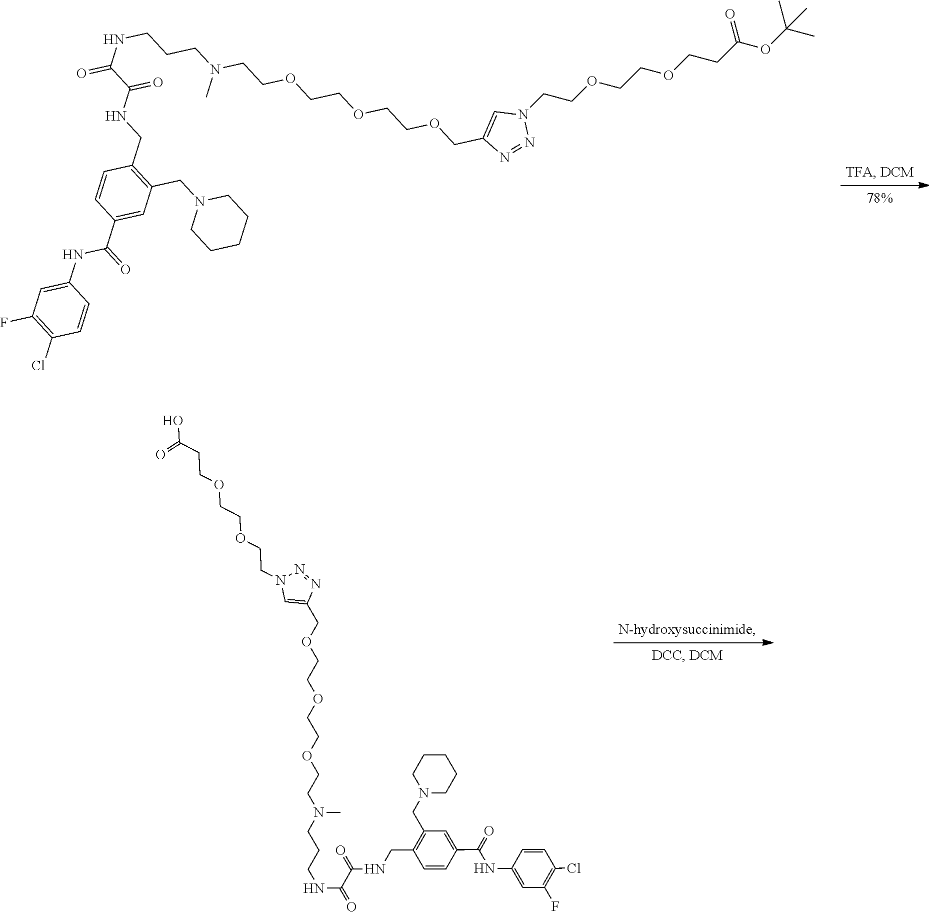 [SMILES: CN(CCCNC(=O)C(=O)NCc1ccc(C(=O)Nc2ccc(Cl)c(F)c2)cc1CN1CCCCC1)CCOCCOCCOCc1cn(CCOCCOCCC(=O)OC(C)(C)C)nn1 ; CN(CCCNC(=O)C(=O)NCc1ccc(C(=O)Nc2ccc(Cl)c(F)c2)cc1CN1CCCCC1)CCOCCOCCOCc1cn(CCOCCOCCC(=O)O)nn1]

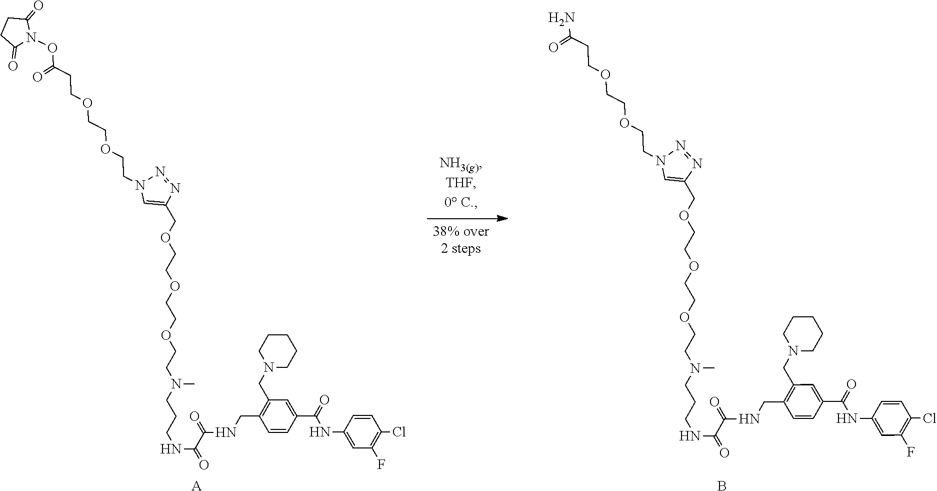

Other conjugations may also be considered, such as e.g., a cysteine conjugation and other site specific conjugation methods. With regard to these various conjugations, a suitable conjugator can be made accordingly with the similar chemistry schemes set forth herein.

### Example 3

### Synthesis of gp160 Attachment Inhibitor

A gp160 attachment inhibitor was made according to the following synthesis route:

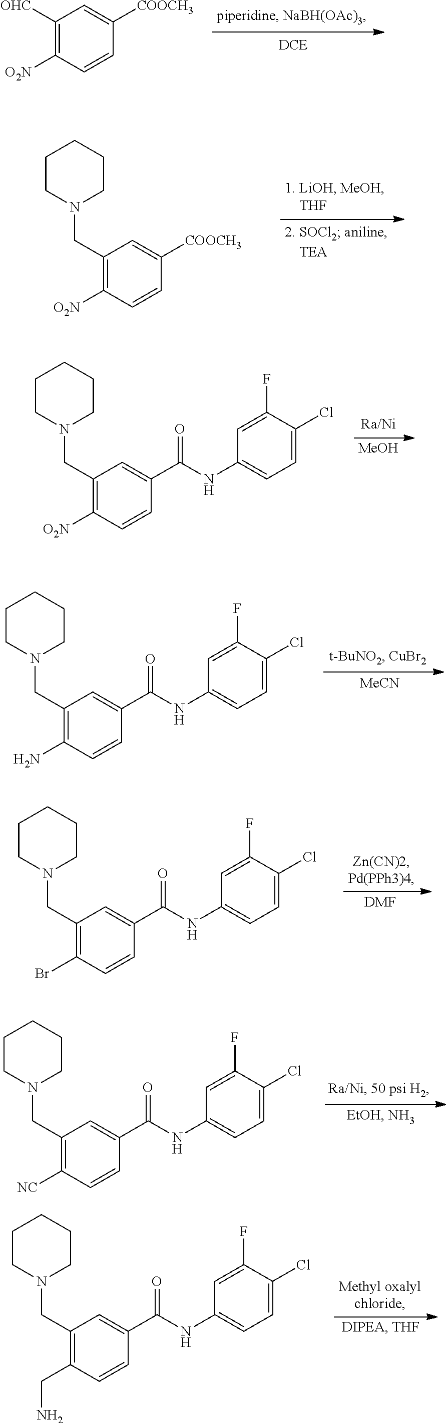

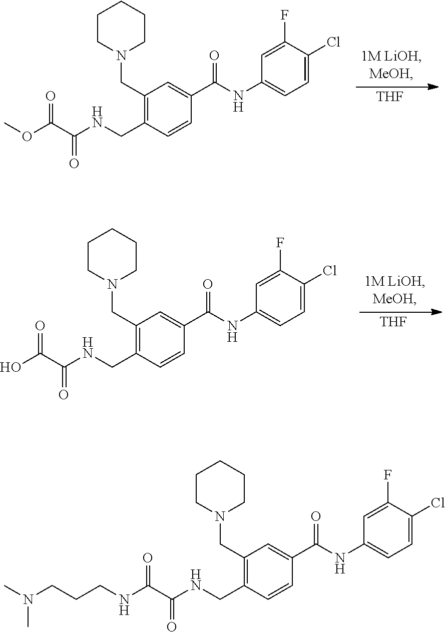 [SMILES: COC(=O)C(=O)NCc1ccc(C(=O)Nc2ccc(Cl)c(F)c2)cc1CN1CCCCC1 ; O=C(O)C(=O)NCc1ccc(C(=O)Nc2ccc(Cl)c(F)c2)cc1CN1CCCCC1 ; CN(C)CCCNC(=O)C(=O)NCc1ccc(C(=O)Nc2ccc(Cl)c(F)c2)cc1CN1CCCCC1 ; "THF" ; "THF"]

### N1-(4((4-chloro-3-fluorophenyl)carbamoyl)-2-(piperidin-1-ylmethy)benzyl)-N2-(3-(dimethylamino)propyl)oxalamide

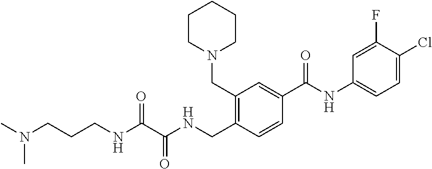 [SMILES: CN(C)CCCNC(=O)C(=O)NCc1ccc(C(=O)Nc2ccc(Cl)c(F)c2)cc1CN1CCCCC1]

### Step 1

### Methyl 4-nitro-3-(piperidin-1-ylmethyl)benzoate

A solution of methyl 3-formyl-4-nitrobenzoate (15 g, 71.7 mmol) and piperidine (14.17 mL, 143 mmol) in 1,2-Dichloroethane (DCE) (150 mL) was treated with acetic acid (8.21 mL, 143 mmol). After 30 min the reaction mixture was treated with sodium triacetoxyborohydride (24.32 g, 115 mmol) and stirred overnight. The reaction was quenched with sat. NaHCO₃, extracted with DCM, washed with sat NaHCO₃, brine, dried with Na₂SO₄, filtered, and concentrated. The residue was purified by silica gel chromatography (EtOAc/Hexane gradient) to afford methyl 4-nitro-3-(1-piperidinylmethyl)benzoate (16.14 g, 58.0 mmol, 81 yield). LC/MS (m/z) ES+=279.3 (M+1)+

### Step 2

### N-14-chloro-3-fluorophenyl)-4-nitro-3-(piperidin-1-ylmethyl)benzamide

A solution of methyl 4-nitro-3-(1-piperidinylmethyl)benzoate (16.05 g, 57.7 mmol) in Tetrahydrofuran (THF) (100 mL) and methanol (100 mL) was treated with LiOH (250 mL, 250 mmol) and stirred at ambient temperature for 4 hours. The mixture was concentrated to give crude 4-nitro-3-(1-piperidinylmethyl)benzoic acid (20.51 g). The acid intermediate was suspended in SOCl₂ (50 mL, 685 mmol), refluxed for 1.5 hours, and concentrated to give 4-nitro-3-(1-piperidinylmethyl)benzoyl chloride (LCMS in meoh, ES+279, methyl ester). The acyl chloride was suspended in dichloromethane (DCM) (100 mL), treated with 4-chloro-3-fluoroaniline (7.97 g, 54.8 mmol), Et₃N (12.06 mL, 87 mmol), and stirred at ambient temperature overnight. Additional Et₃N (4 mL), DCM (11 mL), and aniline (418 mg) was added, and the reaction was stirred overnight. The suspension was quenched with sat. NaHCO₃, extracted with DCM 2x, washed with sat. NaHCO3 1×, Brine, dried with Na2SO4, filtered, and concentrated. Purification by silica gel column chromatography (0-50% EtOAc/Hexane) gave N-(4-chloro-3-fluorophenyl)-4-nitro-3-(1-piperidinylmethyl)benzamide (13.86 g, 35.4 mmol, 61.3% yield) as yellow solid. LC/MS (m/z) ES+=279.3 (M+1)+

### Step 3

### 4-amino-N-14-chloro-3-fluorophenyl)-3-(piperidin-1-ylmethyl)benzamide

A solution of N-(4-chloro-3-fluorophenyl)-4-nitro-3-(1-piperidinylmethyl)benzamide (13 g, 33.2 mmol) in methanol (130 mL) was added slowly to a refluxing mixture of hydrazine hydrate (16.14 mL, 332 mmol) and raney 2800 nickel (4.2 g, 33.2 mmol) in Methanol (130 mL). The reaction was refluxed for 1 hour, cooled to ambient temperature, filtered through celite, washed with MeOH and DCM, and then concentrated to give crude 4-amino-N-(4-chloro-3-fluorophenyl)-3-(1-piperidinylmethyl)benzamide (11.56 g, 31.9 mmol, 96% yield) as light yellow solid. LC/MS (m/z) ES+=362.3 (M+1)+

### Step 4

### 4-bromo-N-14-chloro-3-fluorophenyl)-3-(piperidin-1-ylmethyl)benzamide

An ice cold mixture of copper(II) bromide (0.648 g, 2.90 mmol) in acetonitrile (20 mL) was treated with t-butylnitrite (0.730 mL, 5.53 mmol) followed by 4-amino-N-(4-chloro-3-fluorophenyl)-3-(1-piperidinylmethyl)benzamide (1.000 g, 2.76 mmol) and the resulting dark mixture was stirred overnight at ambient temperature. Saturated NaHCO₃ was added and diluted with ethyl acetate. The mixture was filtered through a pad of Celite and the aq. layer extracted with EA. The extracts were washed with brine, dried over Na₂SO₄, filtered and concentrated. The residue was purified by silica gel chromatography (0-10% MeOH/DCM gradient) to give a dark residue (482 mg, 32%). 1H NMR (400 MHz, METHANOL-d4) δ ppm 1.50 (d, J=5.07 Hz, 2H), 1.56-1.71 (m, 4H), 2.53 (br. s., 4H), 3.67 (s, 2H), 7.37-7.48 (m, 2H), 7.68-7.76 (m, 2H), 7.78-7.87 (m, 1H), 8.05 (d, J=1.95 Hz, 1H); LC/MS (m/z) ES+=425 (M+1).

### Step 5

### N-14-chloro-3-fluorophenyl)-4-cyano-3-(piperidin-1-ylmethyl)benzamide

A suspension of 4-bromo-N-(4-chloro-3-fluorophenyl)-3-(1-piperidinyl methyl)benzamide (2 g, 4.70 mmol) and Zn(CN)₂ (0.386 g, 3.29 mmol) in N,N-Dimethylformamide (DMF) (23.49 ml) was degassed for 5 min with N2 and then treated with Pd(PPh₃)₄ (0.271 g, 0.235 mmol). The reaction mixture was irradiated in the microwave for 20 min @ 120° C. The reaction mixture was poured into water and extracted with EtOAc. The combined extracts were washed with brine, dried (Na₂SO₄), filtered and concentrated. The residue was purified by silica gel chromatography (0-50% EtOAc-hexanes) to afford N-(4-chloro-3-fluorophenyl)-4-cyano-3-(1-piperidinylmethyl)benzamide (1.66 g, 4.46 mmol, 95 yield). LC/MS (m/z) ES+=372.3 (M+1).

### Step 6

### 4-(aminomethyl)-N-(4-chloro-3-fluorophenyl)-3-(piperidin-1-ylmethyl)benzamide

A mixture of N-(4-chloro-3-fluorophenyl)-4-cyano-3-(1-piperidinylmethyl)benzamide (5.00 g, 13.45 mmol) in Ethanol (100 mL) saturated with ammonia gas was treated with Raney 2800 nickel (12 mL, 13.45 mmol) and then stirred under 50 psi hydrogen gas for 72h. The mixture was filtered over Celite, washed with ethyl acetate, DCM, and MeOH. The filtrate was concentrated to give the title compound product as a light green tinged solid. ¹H NMR (400 MHz, METHANOL-d4) δ ppm 1.56 (br. s., 6H), 2.48 (br. s., 4H), 3.61 (s, 2H), 3.90 (s, 2H), 7.38-7.55 (m, 3H), 7.76-7.90 (m, 3H); LC/MS (m/z) ES+=376 (M+1).

### Step 7

### methyl 2((4-((4-chloro-3-fluorophenyl)carbamoyl)-2-(piperidin-1-ylmethyl)benzyl)amino)-2-oxoacetate

An ice cold mixture of 4-(aminomethyl)-N-(4-chloro-3-fluorophenyl)-3-(1-piperidinylmethyl)benzamide (1.000 g, 2.66 mmol) and Hunig's base (0.697 mL, 3.99 mmol) in Tetrahydrofuran (THF) (20 mL) was slowly treated with methyloxalyl chloride (0.270 mL, 2.93 mmol). The mixture was stirred for 5 minutes and judged complete by LCMS. The mixture was diluted with ethyl acetate, washed with sat′d NaHCO₃, then brine, dried over Na₂SO₄, filtered and concentrated. The residue was purified by silica gel chromatography (0-10% MeOH/DCM) to give the title compound as a pale yellow solid. 1H NMR (400 MHz, DMSO-d6) δ ppm 1.33-1.46 (m, 2H), 1.46-1.57 (m, 4H), 2.37 (br. s., 4H), 3.57 (s, 2H), 3.78 (s, 3H), 4.56 (d, J=6.06 Hz, 2H), 7.43 (d, J=8.01 Hz, 1H), 7.51-7.62 (m, 2H), 7.80 (d, J=1.56 Hz, 1H), 7.85 (dd, J=8.01, 1.76 Hz, 1H), 7.91-7.97 (m, 1H), 9.51 (t, J=6.06 Hz, 1H), 10.49 (s, 1H); LC/MS (m/z) ES+=462 (M+1).

### Step 8

### 2-((4-((4-chloro-3-fluorophenyl)carbamoyl)-2-(piperidin-1-ylmethyl)benzyl)amino)-2-oxoacetic acid

A solution of methyl 2-((4-((4-chloro-3-fluorophenyl)carbamoyl)-2-(piperidin-1-ylmethyl)benzyl)amino)-2-oxoacetate (288 mg, 0.623 mmol) in MeOH (5 mL) and THF (5 mL) and treated with 1 M LiOH (1 mL). After 2h the reaction mixture was concentrated in vacuo to afford the title compound (279 mg, 106%). LC/MS (m/z) ES+=448.3 (M+1).

### Step 9

### N1-(4-((4-chloro-3-fluorophenyl)carbamoyl)-2-(piperidin-1-ylmethyl)benzyl)-N2-(3-(dimethylamino)propyl)oxalamide

A mixture of ({[4-{[(4-chloro-3-fluorophenyl)amino]carbonyl}-2-(1-piperidinylmethyl)phenyl] methyl}amino)(oxo)acetic acid (30.0 mg, 0.066 mmol), N,N-dimethyl-1,3-propanediamine (0.017 mL, 0.132 mmol) and Hunig's base (0.035 mL, 0.198 mmol) in N,N-Dimethylformamide (DMF) (1.0 mL) was treated with T3P (0.079 mL, 0.132 mmol) and then stirred at ambient temperature for 5 minutes. The mixture was purified by RP-HPLC (TFA modified) to give slightly impure desired product which was further purified by RP-HPLC (NH4OH modified) to give the desired product as a white solid. 1H NMR (400 MHz, DMSO-d6) δ ppm 1.40 (br. s., 2H), 1.49-1.64 (m, 6H), 2.09 (s, 6H), 2.18 (t, J=7.02 Hz, 2H), 2.37 (br. s., 4H), 3.15 (q, J=6.57 Hz, 2H), 3.57 (s, 2H), 4.52 (d, J=6.24 Hz, 2H), 7.42 (d, J=7.80 Hz, 1H), 7.52-7.66 (m, 2H), 7.79 (s, 1H), 7.84 (dd, J=7.90, 1.46 Hz, 1H), 7.89-8.00 (m, 1H), 8.85 (t, J=5.95 Hz, 1H), 9.23-9.36 (m, 1H), 10.48 (s, 1H); LC/MS (m/z) ES+=532 (M+1).

### Examples 4-8

### Preparation of Antibody-Drug-Conjugates

Antibody drug conjugates as set forth below were made as set forth below; Experimental materials:

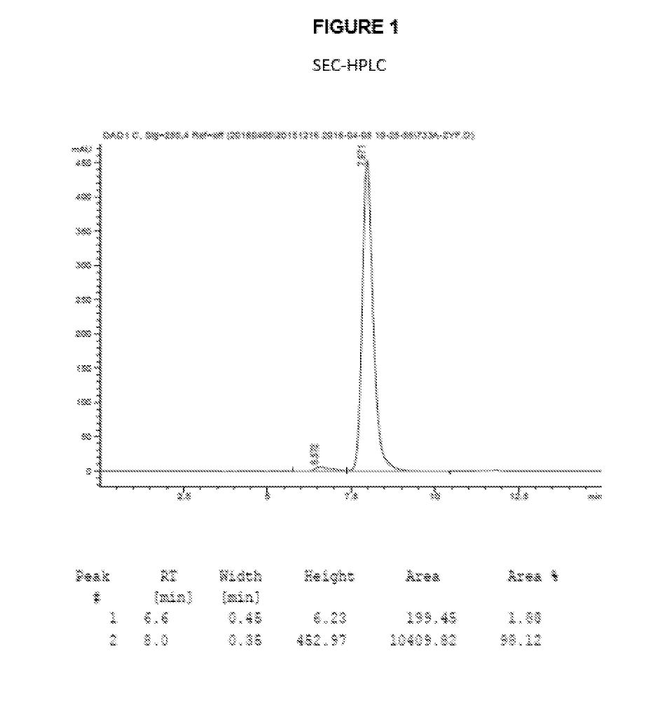

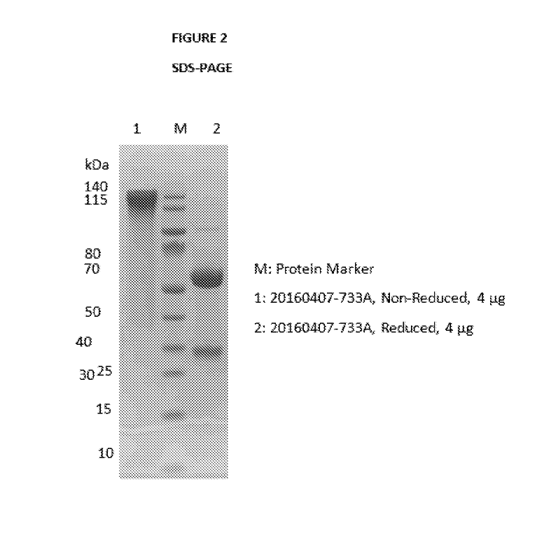

VRC01 was expressed in CHO cells. Cell culture supernatants were collected and purified with a Protein A column and SEC column. The broadly neutralizing antibody VRC01 was stored in 20 mM Histidine buffer (with 5% sucrose, pH 6.0). The purity was confirmed by size exclusion chromatography (SEC-HPLC, FIG. 1) analysis and sodium dodecyl sulfate polyacrylamide gel electrophoresis (SDS-PAGE, FIG. 2).

TABLE 3

| | |
|---|---|
| WBP Code | 733 |
| Product Lot # | 20160407-733A |
| Molecule Type | IgG1 |
| Client ID | VRC01 |
| Cell Lines | CHO |
| Expression Scientist | |
| Fermentation Volume | 5 L |
| Harvest Days | D 11 |
| Formulation | 20 mM histidine, 5% sucrose, pH 6.0 |
| Purity by Reduced SDS-PAGE | 97.8% |
| Purity by SEC-HPLC | 98.12% |
| Endotoxin (LAL) | <1 EU/mg |
| Protein Concentration | 10.76 mg/mL |
| Volume | 21.88 mL |

Four different payload-linkers (PLs) used for the conjugation were designed and made as follows:

Compound #1 (Payload A, compound LA)

Compound #2 (Payload A, compound SA)

Compound #3 (Payload B, compound SB)

Compound #4 (Payload B, compound LB)

Wherein:

LA: long linker payload A

SA: short linker payload A

SB: short linker payload B

LB: long linker payload B

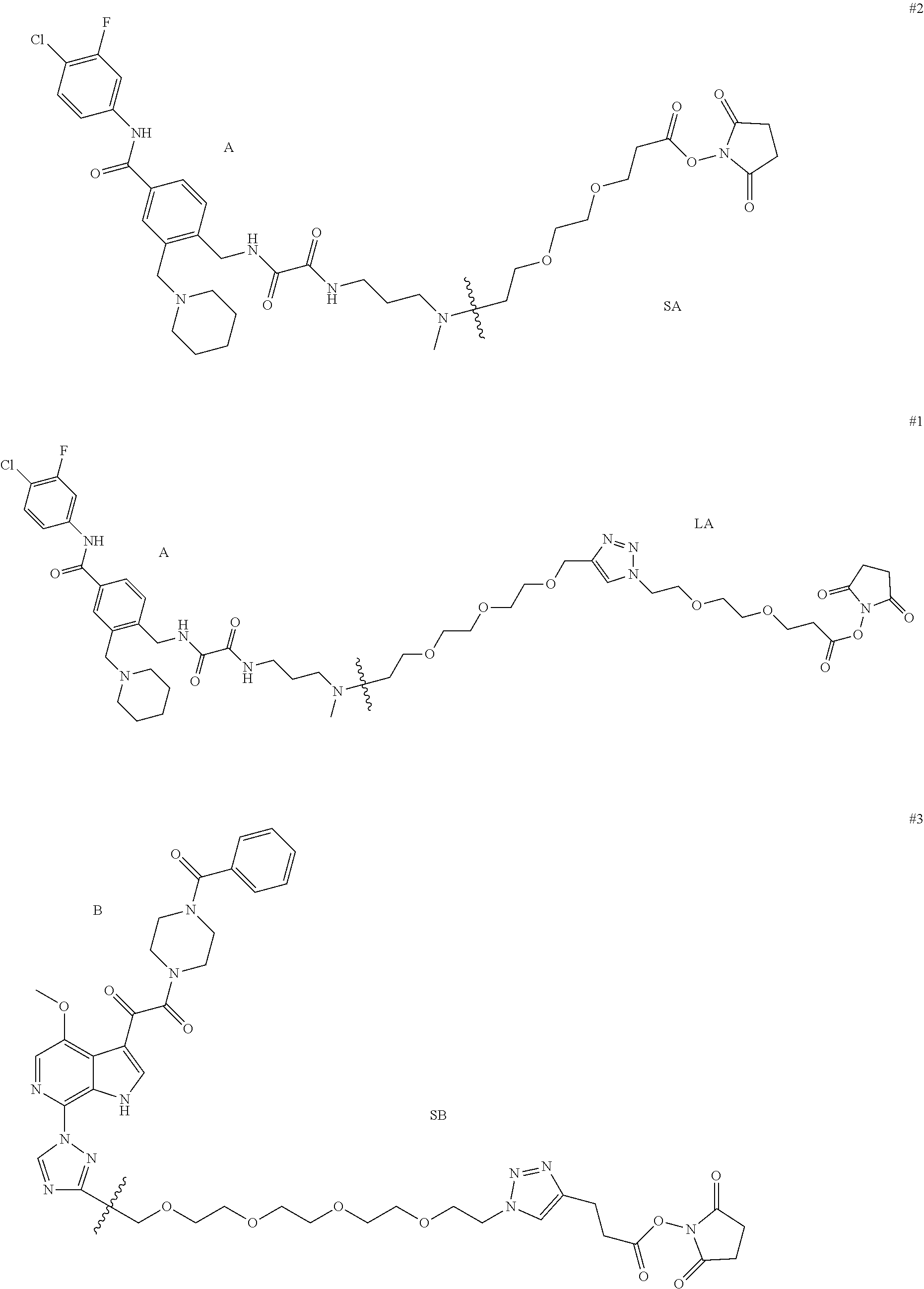

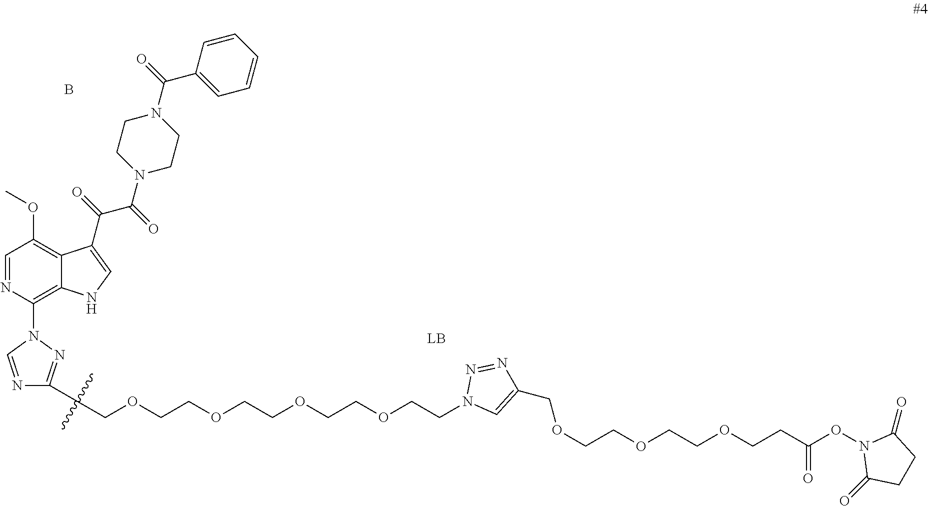

### Analytical Methods

### HPLC Methods

### SEC Analytical Method

TABLE 4

| Column | TOSON - G3000SW_{XL} No. 008541 | |
|---|---|---|
| Column oven temp. | 22° C. | |
| Wave length | 280 nm | |
| Load volume | 10~20 μL | |
| Mobile phase | 0.2M K₂HPO₄/KH₂PO₄, | |
| | 0.25M KCl, 15% (v/v) 2-propanol, pH 7.0 | |
| Gradient Program | time (min) | Flow rate (mL/min) |
| | 0.0 | 0.75 |
| | 18.0 | 0.75 |

Free Drug Assay is set forth in Table 5, with a write-up presented in Example 4.

TABLE 5

| Column: | Supelco HISEP, 4.6*250 mm, 5 μm, | | | |
|---|---|---|---|---|
| | CJ-00002975 | | | |
| Detection Wavelength: | 315 nm & 260 nm | | | |
| Column Oven Temp.: | 30° C. | | | |
| Sampler Temp.: | 4° C. | | | |
| Stop Time: | 45 min | | | |
| Injection Quality: | 60 μg or 20 μL | | | |
| Mobile Phases: | Mobile Phase A: 100 mM Ammonium Acetate | | | |
| | Mobile Phase B: 100% acetonitrile | | | |
| Gradient Program: | Time (min) | A (%) | B (%) | Flow (mL/min) |
| | 0.0 | 75 | 25 | 0.5 |
| | 25.0 | 60 | 40 | 0.7 |
| | 27.0 | 0 | 100 | 0.7 |
| | 29.0 | 0 | 100 | 0.7 |
| | 29.5 | 75 | 25 | 0.7 |
| | 30.0 | 75 | 25 | 1.0 |
| | 45.0 | 75 | 25 | 1.0 |

UV method to determine DAR: UV/Vis and SEC (UV detector) base on Beer-Lambert Law A=E*c*l
A280=E^{mAb}₂₈₀*[mAb]*l+E^{PL}₂₈₀*[PL]*l
A315=E^{mAb}₃₁₅*[mAb]*l+E^{PL}₃₁₅*[PL]*l
[mAb]: mAb concentration
PL: payload-linker
[PL]: payload-linker concentration
E: molar extinction coefficient
c: concentration
l: light path (Nanodrop: 0.1 cm)

### Example 4

The solution of compound SB in dimethylacetamide (DMA, 10 mg/mL) was prepared by dissolving 1.2 mg compound SB (Compound #3) in 0.12 mL DMA). The solution of compound LB in DMA (10 mg/mL) was prepared by dissolving 2.1 mg compound LB (Compound #4) in 0.21 mL DMA. The solution of compound LA in acetonitrile (ACN, 10 mg/mL) was prepared by dissolving 1.7 mg compound LA in 0.17 mLACN. The solution of compound SA in ACN (10 mg/mL) was prepared by dissolving 1.3 mg compound SA (Compound #2) in 0.13 mL ACN.

To determine the retention times of payload-linkers, the solution of LA, SA and LB, SB prepared above were diluted with dilution buffer (50% 100 mM NH₄OAc+50 acetonitrile) to 1 mg/mL. The LA, SA and LB, SB all showed two peaks in HPLC (parent O—Su and hydrolyzed —COOH). The final antibody-drug conjugates (ADCs) samples were submitted to HPLC (Mix model RP column) to determine the free payload-linker level. All ADC products had a distinguish peak at 3.3 min (antibody related), and no other peak appeared in the spectrum. The results showed that the remaining free payload-linker concentrations in the ADC solutions were below the detection limit.

In order to determine the free payload-linker detection limit, different concentrations of LA, SA and LB, SB were prepared and they were submitted to HPLC (Mix model RP column), and the results showed that the detection limits for all 4 payload-linkers are less than 0.0024 pg/mL.

Drug antibody ratio (referenced herein as “DAR”) MS

### Example 5

### Sample Preparation

100 μg protein sample was added to a 1.5 mL tube, thus making up to 100 μL with 2 μL 1 mol/L Tris-HCl buffer, 2.5 μL PNGase F solution and Milli-Q water. This was mixed well and incubated at 37° C. for 4 hours.

400 μL 50 mM sodium phosphate buffer was added into the sample using an ultra-filtration tube, followed by centrifuging at 13000 rpm for 15 min. Then the sample was transferred to a 1.5 mL tube, 50 mM of sodium phosphate was added to a final volume of 100 μL. 1 μL of sialidase A and 2 μL O-glycanase was added, and incubated at 37° C. for 2 hours.

TABLE 6
HPLC conditions

| Column | Agilent, PLRP-S 1000A 2.1 × 50 mm, 8 μm | | | |
|---|---|---|---|---|
| Column oven temp. | 80° C. | | | |
| Sample temp. | 4° C. | | | |
| Wave length | 280 nm | | | |
| Load volume | 5 μL | | | |
| Mobile phase | Mobile Phase A: 0.05% TFA in water | | | |
| | Mobile Phase B: 0.04% TFA in CAN | | | |
| Gradient Program | Time (min) | % A | % B | Flow Rate (mL/min) |
| | 0.0 | 95 | 5 | 0.4 |
| | 0.1 | 95 | 5 | 0.4 |
| | 0.5 | 70 | 30 | 0.4 |
| | 2.0 | 70 | 30 | 0.4 |
| | 5.0 | 58 | 42 | 0.4 |
| | 5.5 | 58 | 42 | 0.4 |
| | 5.6 | 5 | 95 | 0.4 |
| | 6.4 | 5 | 95 | 0.4 |
| | 6.5 | 95 | 5 | 0.4 |
| | 8.0 | 95 | 5 | 0.4 |

TABLE 7
MS conditions

| | |
|---|---|
| Ion Polarity | Positive |
| Data Storage | Profile |
| Gas Temp. | 325° C. (Depend on specific sample) |
| Drying Gas | 12 L/min (Depend on specific sample) |
| Nebulizer | 55 psig (Depend on specific sample) |
| Capillary Voltage | 3500 V (Depend on specific sample) |
| Fragmentor | 300 V (Depend on specific sample) |
| Skimmer | 65 V (Depend on specific sample) |
| OCT1 RF Vpp | 750 V (Depend on specific sample) |
| Acquisition Mode | MS (seg) (Depend on specific sample) |
| Mass Range | 500-8000 m/z (Depend on specific sample) |
| Acquisition Rate | 1 spectra/s (Depend on specific sample) |
| Acquisition Time | 0.5-7.0 min (Depend on specific sample) |
| Analyzer Mode | Extended Mass Range (20000 m/z) |

### Example 6

### Preparation of VRC01-LA and VRC01-SA

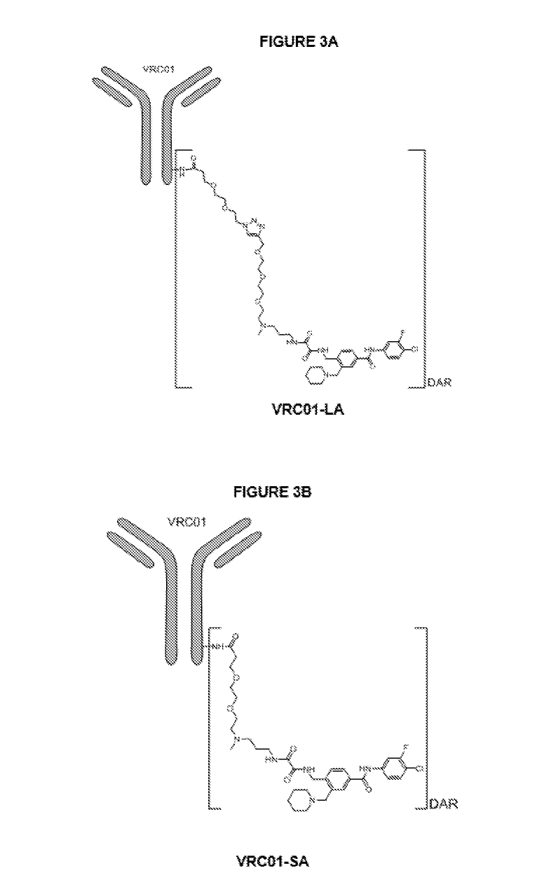

Reaction set up (LA and SA): VRC01-LA and VRC01-SA, referred to in FIGS. 3A and 3B respectively, VRC01-LA and VRC01-SA were prepared. CH₃CN was added into VRC01 solution in PBS buffer (pH 7.5) and the reaction was mixed before the addition of LA or SA solution in CH₃CN. Total CH₃CN content in conjugation solution was 20% after the addition of payload-linkers. The reaction mixture was then placed in a shocker (150 rounds per minute) inside a 22° C. incubator for two hours. After two hours, the reaction mixture was taken out and subjected into buffer exchange to storage buffer and free payload-linker removal by using spin desalting column and amicon ultrafiltration (30 kDa). About 20-25 mg final products were obtained and the reaction conversions were around 60% and 90%.

TABLE 8

| CmAb | Conj. | Conj. | CH₃CN | C drug in | Conj. | Storage | Scale |
|---|---|---|---|---|---|---|---|
| (mg/mL) | Temp | time | (%) | CH₃CN | Buffer | Buffer | (mg) |
| 10 | 22° C. | 2 h | 20 | LA: 7.63 | PBS | 20 mM His, | 30 |
| | | | | mg/mL | pH 7.5 | 5% Sucrose | |

| | | UV | SEC | MS DAR | | Aggregate | D 0 |
|---|---|---|---|---|---|---|---|
| Entry | LA eq | DAR | DAR | de-N | de-N,O | (%) | (%) |
| VRC01-LA | 18 | 2.28 | 2.01 | NA | 3.4 | 1.83 | 4.81 |

* 30 ma VRC01-LA generation

| CmAb | Conj. | Conj. | CH₃CN | Cdrug in | Conj. | Storage | Scale |
|---|---|---|---|---|---|---|---|
| (mg/mL) | Temp | time | (%) | CH₃CN | Buffer | Buffer | (mg) |
| 10 | 22° C. | 2 h | 20 | SA: 7.84 | PBS | 20 mM His, | 30 |
| | | | | mg/mL | pH 7.5 | 5% Sucrose | |

| | | UV | SEC | MS DAR | | Aggregate | D 0 |
|---|---|---|---|---|---|---|---|
| Entry | SA eq | DAR | DAR | de-N | de-N,O | (%) | (%) |
| VRC01-SA | 27 | 1.62 | 1.28 | NA | 3.0 | 2.07 | 5.02 |

*30 mg VRC01-SA generation

### Example 7

### Preparation of VRC01-LB and VRC01-SB

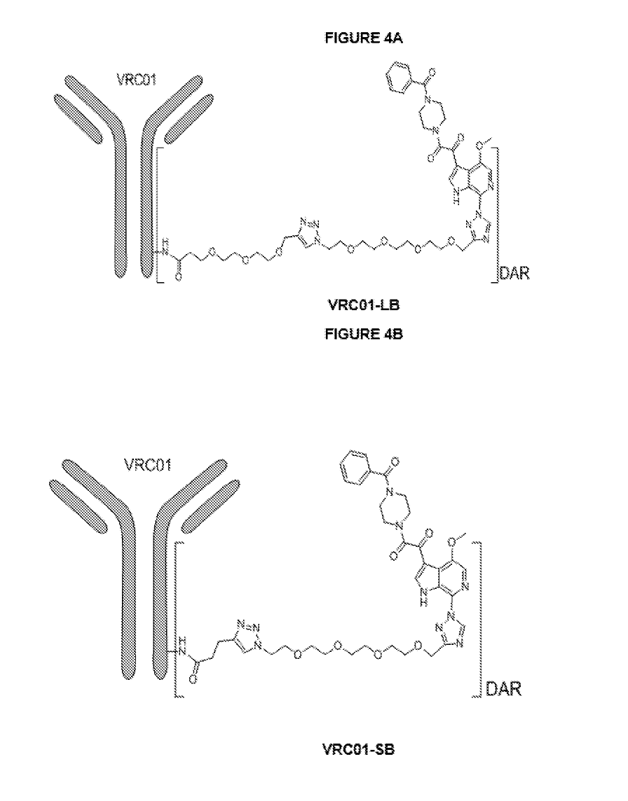

Reaction set up (LB, SB): VRC01-LB and VRC01-SB, referred to in FIGS. 4A and 4B respectively, DMA was added into a VRC01 solution in PBS buffer (pH 7.5) and the reaction was mixed properly before the addition of LB or SB solution in DMA. Total DMA content in conjugation solution was 10% after the addition of payload-linkers. The reaction mixture was then placed in a shocker (150 rounds per minute) inside a 22° C. incubator for two hours. After two hours, the reaction mixture was taken out and subjected to buffer exchange to storage buffer and free drug removal by using spin desalting column. About 20-25 mg final products were obtained and the reaction conversion rates were around 70% and 80%.

TABLE 9

| CmAb | Conj. | Conj. | DMA | Cdrug in | Conj. | Storage | Scale |
|---|---|---|---|---|---|---|---|
| (mg/mL) | Temp | time | (%) | DMA | Buffer | Buffer | (mg) |
| 10 | 22° C. | 2 h | 10 | 10 | PBS | 20 mM His, | 25 (SB) |
| | | | | mg/mL | pH 7.5 | 5% Sucrose | 30 (LB) |

| | | UV | SEC | MS DAR | | Aggregate | D 0 |
|---|---|---|---|---|---|---|---|
| Entry | PL eq | DAR | DAR | de-N | de-N,O | (%) | (%) |
| VRC01-SB | 6.0 | 5.28 | 8.53 | NA | 3.9 | 2.02 | 3.49 |
| VRC01-LB | 6.0 | 5.18 | 7.86 | NA | 3.8 | 1.97 | 4.65 |

*25-30 mg VRC01-LB and SB generation;
PL: payload-linker

### Free Payload-Linker Removal

A total 4 ADC products (1.9 mL of VRC01-SB, 2.1 mL of VRC01-LB, 1.65 mL of VRC01-LA and 1.15 mL of VRC01-SA) in dialysis cassette (0.5-3 mL capacity, MWCO: 10,000) were respectively dialyzed against 500 mL of the buffer (20 mM histidine, pH 6.0) six times to remove free payload-linkers. After dialysis, the concentration, free drug content and endotoxin of the resulting ADC products were determined by UV/Vis, HPLC (Mix model RP column) and Microplate Reader. Then 5% sucrose was respectively added into the ADC solutions. The DARs and the aggregate contents of the ADC products were determined by SEC-HPLC.

TABLE 10
Characterizations of all ADCs (no free payload-
linker was detected: <0.0024 μg/mL)

| | UV- | SEC- | Aggregate | Concentration | Endotoxin |
|---|---|---|---|---|---|
| Compound ID | DAR | DAR | (%) | (mg/mL) | (EU/mg) |
| VRC01-SB | 5.60 | 8.43 | 2.53 | 4.93 | 0.133 |
| VRC01-LB | 5.11 | 7.79 | 2.09 | 5.98 | 0.129 |
| VRC01-LA | 2.60 | 1.71 | 1.17 | 4.90 | 0.163 |
| VRC01-SA | 2.20 | 1.29 | 1.95 | 6.41 | 0.052 |

### Example 8

### Preparation of VRC01-LA-SB and VRC01-LA-LB

Reaction Set Up:

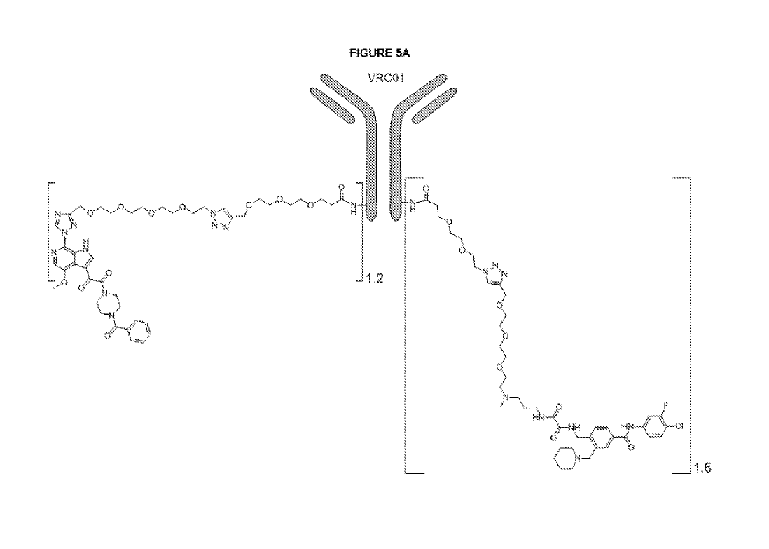

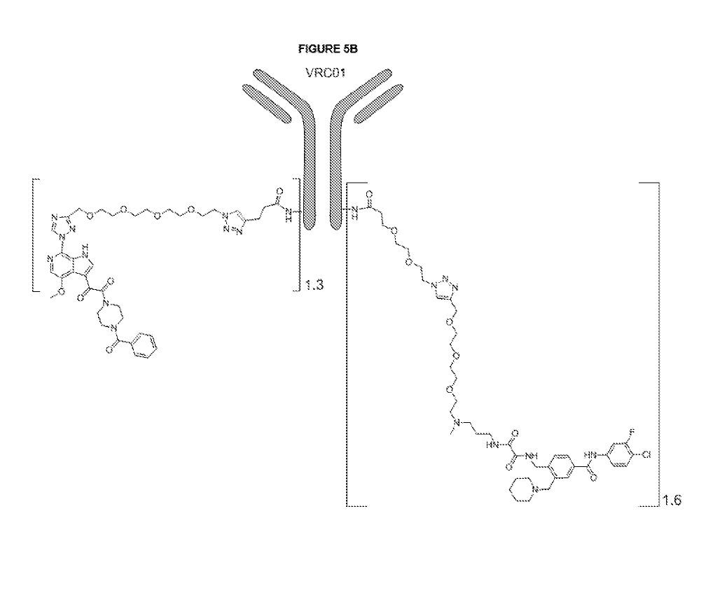

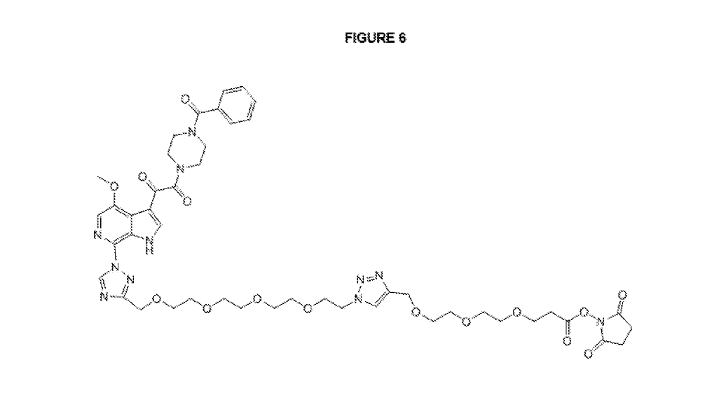

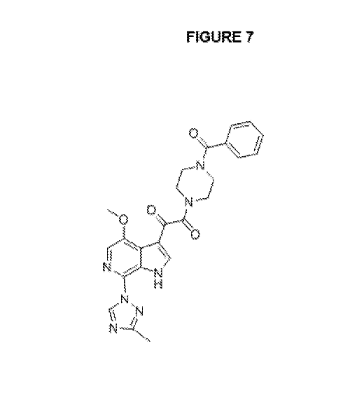

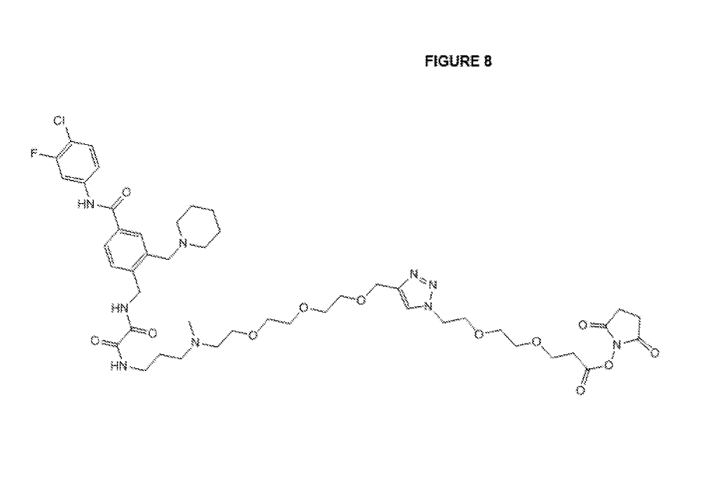

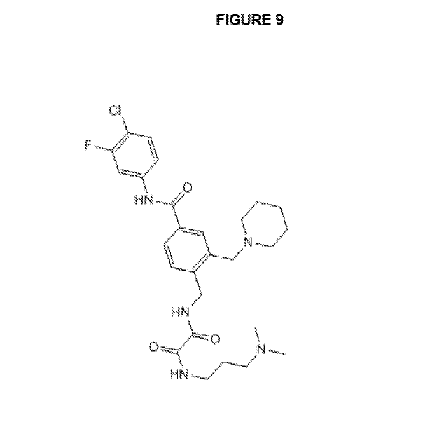

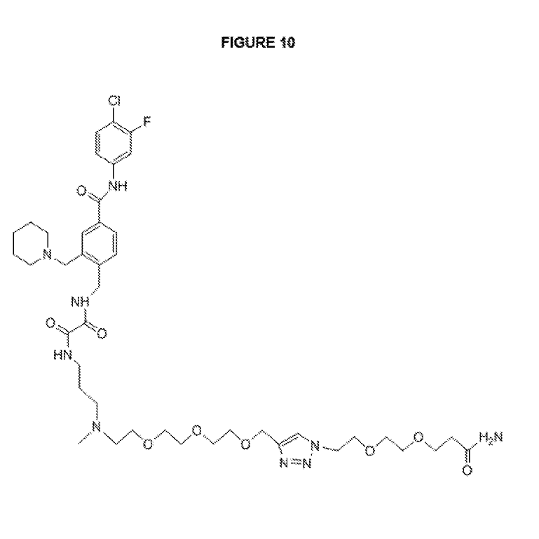

Final structure products resulting from the synthesis below are referenced in FIGS. 5A and 5B. CH₃CN was added into VRC01 solution in PBS buffer (pH 7.5) and the reaction was mixed properly before the addition of LA solution in CH₃CN. Total CH₃CN content in conjugation solution was 20% and the final mAb concentration in the reaction was 10 mg/mL. After the addition of payload-linker, the reaction mixture was then placed placed on a rotary platform (10 rounds per min) at 22° C. in an incubator for two hours. Then reaction mixture was taken out and the free payload-linker (LA) was removed by using amicon altrafiltration (50 kDa). About 2 mg of ADC was subjected to intact MS and SEC for DAR measurement and aggregation determination.

The remaining 18 mg reaction mixture was divided into two vials (9.0 mg each) and were submitted to the next conjugations with SB and LB respectively. DMA and SB, LB solutions in DMA were added into the ADC PBS buffer prepared above. DMA content was 20% and the ADC concentration was 10 mg/mL in the conjugation reactions. The conjugation solutions were placed on a rotary platform (10 rounds per min) inside a 22° C. incubator for 2 h. The reaction mixtures were then subjected to buffer exchange to storage buffer and free payload-linker removal by using amicon ultrafiltration (50 kDa). Lastly, the reaction mixtures were dialyzed to further remove free payload-linker to undetectable level and buffer exchange (3 days, 6 times buffer exchanges). About 4.5 mg (each) final products were obtained and the reaction conversion rates were around 50%.

Determination of the Free Drug Level in these ADC Products:

To determine retention times of mAb and free payload-linkers, the solution of LA, LB, and SB (all the concentration of drug were 10 mg/mL) were diluted with dilution buffer (50% 100 mM NH₄Ac+50% acetonitrile) to 0.2 mg/mL. The payload-linker solutions were then mixed with mAb and the final drug concentration and mAb concentration in the samples were 0.1 mg/mL and 1 mg/mL respectively. The LA, LB, and SB showed two peaks in HPLC (Supelco HISEP, 4.6*250 mm, 5 μm, CJ-00005105). When mAb concentration in the sample was 1 mg/mL, there was no peak showing at 3.3 min. When mAb concentration was increased to 3 mg/mL, there was a corresponding peak observed at 3.3 min, which indicated the mAb retention time was 3.3 min.

The ADC samples were subjected to HPLC to determine the free payload-linker level. The ADC products had a peak at 3.3 min, and there is no other peaks were observed, which indicated the free payload-linker concentration in the ADC solution was below detection limit. In order to determine the detection limit, different concentrations of LA, LB, and SB were prepared and subjected to HPLC, and the results showed that the detection limits for LA, LB, and SB were all below 0.006 μg/mL.

TABLE 11
Characterizations of VRC01-LA-SB

| MS- | MS- | Concen- | | | | Aggre- |
|---|---|---|---|---|---|---|
| DAR | DAR | tration | Free drug | | D 0 | gate |
| (LA) | (LB) | (mg/mL) | (%) | Endotoxin | (%) | (%) |
| 1.6 | 1.3 | 4.81 | Below detection | 0.219 | 8.44 | 2.35 |
| | | | limit | EU/mg | | |

* The sample was subjected to de-N- and de-O-glycosylation and then measured by MS to obtain MS DAR.
*Storage buffer: 20 mM histidine, 5% sucrose, pH 6.0

TABLE 12
Characterizations of VRC01-LA-LB

| MS- | MS- | Concen- | | | | Aggre- |
|---|---|---|---|---|---|---|
| DAR | DAR | tration | Free drug | | D 0 | gate |
| (LA) | (LB) | (mg/mL) | (%) | Endotoxin | (%) | (%) |
| 1.6 | 1.2 | 6.56 | Below detection | 0.137 | 5.43 | 2.11 |
| | | | limit | EU/mg | | |

* The sample was subjected to de-N- and de-O-glycosylation and measured by MS to obtain MS DAR.
*Storage buffer: 20 mM histidine, 5% sucrose, pH 6.0

### Biological Data Procedure

A pseudotyped virus assay (PSV) was used to assess the potency of various HIV entry inhibitors. Replication defective virus was produced by co-transfection of a plasmid containing an NL4-3 provirus [containing a mutation in the envelope open reading frame (ORF) and a luciferase reporter gene replacing the nef ORF] and a CMV-promoter expression plasmid containing an ORF for various HIV gp160 envelope clones. The harvested virus was stored at −80C in small aliquots and the titer of the virus measured to produce a robust signal for antiviral assays.

The PSV assay was performed by using U373 cells stably transformed to express human CD4, the primary receptor for HIV entry and either human CXCR4 or human CCR5 which are the co-receptors required for HIV entry as target cells for infection. Molecules of interest (including, but not limited to small molecule inhibitors of HIV, neutralizing antibodies of HIV, antibody-drug conjugate inhibitors of HIV, peptide inhibitors of HIV, and various controls) were diluted into tissue culture media and diluted via serial dilution to create a dose range of concentrations. This dose-range was applied to U373 cells and the pre-made pseudotyped virus added. The amount of luciferase signal produced after 3 days of culture was used to reflect the level of pseudotyped virus infection. An IC50, or the concentration of inhibitor required to reduce PSV infection by 50% from the infection containing no inhibitor was calculated. Assays to measure cytotoxity were performed in parallel to ensure the antiviral activity observed for an inhibitor was distinguishable from reduced target cell viability.

Table 13 provides the materials (i.e., drugs, linkers, antibodies, antibody-drug-conjugates (“ADCs”)) referred to in the results set forth in Tables 14-16 below and Figures detailing the structure of each.

TABLE 13

| Reference No. | Corresponding FIG. |
|---|---|
| 1 | 6 |
| 2 | 7 |
| 3 | 8 |
| 4 | 9 |
| 5 | 10 |
| 6 | 3A |
| 7 | 3B |
| 8 | 4A |
| 9 | 4B |
| 10 | 5A |
| 11 | 5B |

Table 14 provides potency values for drugs and drug-linker materials.

TABLE 14

| Reference No. | | | |
|---|---|---|---|
| (Table 13) | IC50 (μM) | IC50 (μM) | IC50 (μM) |
| 1 | 0.007 | 0.002 | 10 |
| 2 | 0.001 | 0.0002 | 10 |
| 3 | 0.0112 | 0.00017 | 10 |
| 4 | 0.0173 | 0.00017 | 4.25 |
| EFV | 0.0019 | 0.00153 | 0.0016 |

wherein EFV is Efavirenz

Table 15 provides various values for drugs, drug-linker materials and ADCs (mono-payloads).

TABLE 15

| Reference No. | CCIC50 | IC50 | | IC50 | |
|---|---|---|---|---|---|
| (Table 13) | (μM) | (N = 1) | | (N = 2) | |
| VRC01 bnAb for ADC | >100 | >100 | μg/mL | >100 | μg/mL |
| (Refer to seq ID?) | | c.a. 667 | nM | c.a. 667 | nM |
| 2 (Attachment | >100 | 2.9 | nM | 1.7 | nM |
| Inhibitor) | | | | | |
| 1 (Attachment | >100 | 7 | nM | N/A | |
| inhibitor + linker) | | | | | |
| 4 (gp160 inhibitor) | 5.16 | 7.4 | nM | 4.2 | nM |
| 5 (gp160 inhibitor + | >100 | 25 | nM | 7.6 | nM |
| linker) | | | | | |
| 6 (ADC) | >100 | 0.0753 | μg/mL | 0.0413 | μg/mL |
| | | c.a. 0.5 | nM | c.a. 0.27 | nM |
| 7 (ADC) | >100 | 0.4183 | μg/mL | 0.2258 | μg/mL |
| | | c.a. 2.7 | nM | c.a. 1.5 | nM |
| 9 (ADC) | >100 | 0.6169 | μg/mL | 0.7721 | μg/mL |
| | | c.a. 4 | nM | c.a. 5 | nM |
| 8 (ADC) | >100 | 0.5263 | μg/mL | 0.3 | μg/mL |
| | | c.a. 3.4 | nM | c.a. 1.9 | nM |

Table 16 provides various values for drugs, drug-linker materials and ADCs (dual-payloads).

TABLE 16

| Reference No. | CCIC50 | IC50 | | IC50 | |
|---|---|---|---|---|---|
| (Table 13) | (μM) | (N = 1) | | (N = 2) | |
| VRC01 bnAb for ADC | >100 | >100 | μg/mL | >100 | μg/mL |
| (seq ID no?) | | c.a. 667 | nM | c.a. 667 | nM |
| 2 (attachment | >100 | 2.9 | nM | 1.7 | nM |
| inhibitor) | | | | | |
| 1 (attachment | >100 | 7 | nM | N/A | |
| inhibitor + linker) | | | | | |
| 4 (gp160 inhibitor) | 5.16 | 7.4 | nM | 4.2 | nM |
| 5 (gp160inhibitor + | >100 | 25 | nM | 7.6 | nM |
| linker) | | | | | |
| 10 (dual linker ADC) | >100 | 0.1038 | μg/mL | 0.1061 | μg/mL |
| | | c.a. 0.7 | nM | c.a 0.7 | nM |
| 11 (dual linker ADC) | >100 | 0.1542 | μg/mL | 0.1070 | μg/mL |
| | | c.a. 1.0 | nM | c.a. 0.7 | nm |

The present invention is advantageous and offers a contribution to the art. By tethering bNAb and an envelope targeting small molecule via ADC technology, both of which are believed to possess complementary viral coverage profile, a broader viral coverage can be achievable. The pharmacokinetic property of bNAbs (preferably with half-life extension mutations) can be advantageously utilized. Not being bound by theory, HIV treatment with a single bNAb is believed to have an effect on the emergence of resistance. The ADC is capable of possessing multiple antiviral mode of actions (MoAs) all targeting the viral envelope, which may hinder selection of escape variants and improve the resistance profile. The small molecule ARV tethering to the bNAb may have minimal undesired uptake by any other cells/tissues excepting viruses, and this has the ability to improve its safety profile, tolerance, and reduction of effective dose.

In summary, the present invention is highly advantageous in that the antibody-drug-conjugate functions as a bispecific molecule. More specifically, the antibody and the drug, connected via linker, target the HIV envelope employing two distinct and independent mechanisms of action. Accordingly, the invention is unique relative to other antibody-drug-conjugates, and is useful in treating, preventing or curing HIV

### SEQUENCE LISTING

| SEQ | |
|---|---|
| ID | |
| NO | Sequence Description |
| 1 | Amino acid sequence of an example of (gp41) |
| 2 | Amino acid sequence of attachment inhibitor (T-20: |
| | “enfuvirtide”) |
| 3 | VRC01 CDRH1 |
| 4 | VRC01 CDRH2 |
| 5 | VRC01 CDRH3 |
| 6 | VRC01 CDRL1 |
| 7 | VRC01 CDRL2 |
| 8 | VRC01 CDRL3 |
| 9 | VRC01 heavy chain variable region (VH) amino acid sequence |
| 10 | VRC01 light chain variable region (VL) amino acid sequence |
| 11 | VRC01 heavy chain full length amino acid sequence |
| 12 | VRC01-“LS” Fc region heavy chain amino acid sequence |
| 13 | VRC01 light chain full length amino acid sequence |
| 14 | N6 CDRH1 |
| 15 | N6 CDRH2 |
| 16 | N6 CDRH3 |
| 17 | N6 CDRL1 |
| 18 | N6 CDRL2 |
| 19 | N6 CDRL3 |
| 20 | N6 heavy chain variable region (VH) amino acid sequence |
| 21 | N6 light chain variable region (VL) amino acid sequence |
| 22 | N6 heavy chain full length amino acid sequence |
| 23 | N6 light chain full length amino acid sequence |
| 24 | N6-“LS” Fc region heavy chain amino acid sequence |
| 25 | Amino acid sequence of an example of (gp120) |
| 26 | VRC07-523 heavy chain variable region (VH) amino acid sequence |
| 27 | VRC07-523 light chain variable region (VL) amino acid sequence |

SEQ ID NO: 1
AVGIGALFLGFLGAAGSTMGAASMTLTVQARQLLSGIVQQQNNLLRAIEA
QQHLLQLTVWGIKQLQARILAVERYLKDQQLLGIWGCSGKLICTTAVPWN
ASWSNKSLEQIWNHTTWMEWDREINNYTSLIHSLIEESQNQQEKNEQELL
ELDKWASLWNWFNITNWLWYIKLFIMIVGGLVGLRIVFAVLSIVNRVRQG
YSPLSFQTHLPTPRGPDRPEGIEEEGGERDRDRSIRLVNGSLALIWDDLR
SLCLFSYHRLRDLLLIVTRIVELLGRRGWEALKYWWNLLQYWSQELKNSA
VSLLNATAIAVAEGTDRVIEVVQGACRAIRHIPRRIRQGLERILL
SEQ ID NO: 2
Ac-YTSLIHSLIEESQNQQEKNEQELLELDKWASLWNWF-NH₂
SEQ ID NO: 3
DCTLNW
SEQ ID NO: 4
LKPRGGAVNYARPLQG
SEQ ID NO: 5
GKNCDYNWDFEH
SEQ ID NO: 6
RTSQYGSLA
SEQ ID NO: 7
SGSTRAA
SEQ ID NO: 8
QQYEF
SEQ ID NO: 9
QVQLVQSGGQMKKPGESMRISCRASGYEFIDCTLNWIRLAPGKRPEWMGW
LKPRGGAVNYARPLQGRVTMTRDVYSDTAFLELRSLTVDDTAVYFCTRGK
NCDYNWDFEHWGRGTPVIVS
SEQ ID NO: 10
EIVLTQSPGTLSLSPGETAIISCRTSQYGSLAWYQQRPGQAPRLVIYSGS
TRAAGIPDRFSGSRWGPDYNLTISNLESGDFGVYYCQQYEFFGQGTKVQV
DIKRT
SEQ ID NO: 11
QVQLVQSGGQMKKPGESMRISCRASGYEFIDCTLNWIRLAPGKRPEWMGW
LKPRGGAVNYARPLQGRVTMTRDVYSDTAFLELRSLTVDDTAVYFCTRGK
NCDYNWDFEHWGRGTPVIVSSPSTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
SEQ ID NO: 12
QVQLVQSGGQMKKPGESMRISCRASGYEFIDCTLNWIRLAPGKRPEWMGW
LKPRGGAVNYARPLQGRVTMTRDVYSDTAFLELRSLTVDDTAVYFCTRGK
NCDYNWDFEHWGRGTPVIVSSPSTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVLHEALHSHYTQKSLSLSPG
K
SEQ ID NO: 13
EIVLTQSPGTLSLSPGETAIISCRTSQYGSLAWYQQRPGQAPRLVIYSGS
TRAAGIPDRFSGSRWGPDYNLTISNLESGDFGVYYCQQYEFFGQGTKVQV
DIKR
SEQ ID NO: 14
AHILF
SEQ ID NO: 15
WIKPQYGAVNFGGGFRD
SEQ ID NO: 16
DRSYGDSSWALDA
SEQ ID NO: 17
QTSQGVGSDLH
SEQ ID NO: 18
HTSSVED
SEQ ID NO: 19
QVLQF
SEQ ID NO: 20
RAHLVQSGTAMKKPGASVRVSCQTSGYTFTAHILFWFRQAPGRGLEWVGW
IKPQYGAVNFGGGFRDRVTLTRDVYREIAYMDIRGLKPDDTAVYYCARDR
SYGDSSWALDAWGQGTTVVVSA
SEQ ID NO: 21
YIHVTQSPSSLSVSIGDRVTINCQTSQGVGSDLHWYQHKPGRAPKLLIHH
TSSVEDGVPSRFSGSGFHTSFNLTISDLQADDIATYYCQVLQFFGRGSRL
HIK
SEQ ID NO: 22
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCARD
RSYGDSSWALDAWGQGTTVVVSAASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGK
SEQ ID NO: 23
YIHVTQSPSSLSVSIGDRVTINCQTSQGVGSDLHWYQHKPGRAPKLLIHH
TSSVEDGVPSRFSGSGFHTSFNLTISDLQADDIATYYCQVLQFFGRGSRL
HIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSP
VTKSFNRGEC
SEQ ID NO: 24
RAHLVQSGTAMKKPGASVRVSCQTSGYTFTAHILFWFRQAPGRGLEWVG
WIKPQYGAVNFGGGFRDRVTLTRDVYREIAYMDIRGLKPDDTAVYYCARD
RSYGDSSWALDAWGQGTTVVVSAASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVLHEALHSHYTQKSLSLS
PGK
SEQ ID NO: 25
KLWVTVYYGVPVWKEATTTLFCASDAKAYDTEVHNVWATHACVPTDPNPQ
EVVLVNVTENFNMWKNDMVEQMHEDIISLWDQSLKPCVKLTPLCVSLKCT
DLKNDTNTNSSSGRMIMEKGEIKNCSFNISTSIRGKVQKEYAFFYKLDII
PIDNDTTSYKLTSCNTSVITQACPKVSFEPIPIHYCAPAGFAILKCNNKT
FNGTGPCTNVSTVQCTHGIRPVVSTQLLLNGSLAEEEVVIRSVNFTDNAK
TIIVQLNTSVEINCTRPNNNTRKRIRIQRGPGRAFVTIGKIGNMRQAHCN
ISRAKWNNTLKQIASKLREQFGNNKTIIFKQSSGGDPEIVTHSFNCGGEF
FYCNSTQLFNSTWFNSTWSTEGSNNTEGSDTITLPCRIKQIINMWQKVGK
AMYAPPISGQIRCSSNITGLLLTRDGGNSNNESEIFRPGGGDMRDNWRSE
LYKYKVVKIEPLGVAPTKAKRRVVQREKR
SEQ ID NO: 26
QVRLSQSGGQMKKPGDSMRISCRASGYEFINCPINWIRLAPGKRPEWMGW
MKPRHGAVSYARQLQGRVTMTRDMYSETAFLELRSLTSDDTAVYFCTR
GKYCTARDYYNWDFEHWGQGTPVTVSS
SEQ ID NO: 27
SLTQSPGTLSLSPGETAIISCRTSQYGSLAWYQQRPGQAPRLVIYSGSTR
AAGIPDRFSGSRWGPDYNLTISNLESGDFGVYYCQQYEFFGQGTKVQVDI
K

1. An antibody-drug conjugate of the Formula (I):
Ab-L-D (I)
wherein:
Ab comprises a broadly neutralizing antibody having a binding affinity for an HIV envelope glycoprotein;
L comprises a linker molecule covalently bonded to said broadly neutralizing antibody; and
D comprises one or more drugs covalently bonded to said linker molecule, said one or more drugs capable of binding to said HIV envelope glycoprotein.

2. The antibody-drug conjugate according to claim 1, wherein the broadly neutralizing antibody binds to the CD4 binding site, the gp120-gp41 interface, or the gp41 membrane-proximal external region (MPER).

3.-11. (canceled)

12. The antibody-drug conjugate according to claim 1, wherein the linker molecule is a non-cleavable linker.

13. (canceled)

14. The antibody-drug conjugate according to claim 1, wherein the one or more drugs is an attachment inhibitor.

15. The antibody-drug conjugate according to claim 1, wherein the one or more drugs is a compound of the formula:

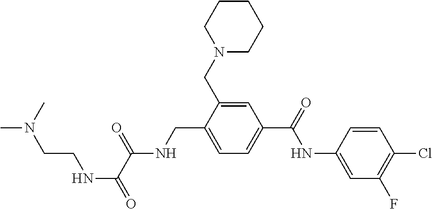 [SMILES: CN(C)CCNC(=O)C(=O)NCc1ccc(C(=O)Nc2ccc(Cl)c(F)c2)cc1CN1CCCCC1]

16. The antibody-drug conjugate according to claim 1, wherein the one or more drugs is a compound of the formula:

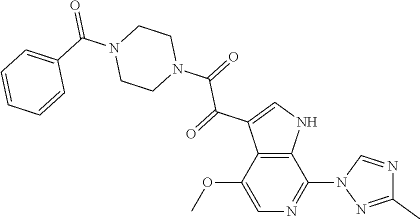 [SMILES: COc1cnc(-n2cnc(C)n2)c2[nH]cc(C(=O)C(=O)N3CCN(C(=O)c4ccccc4)CC3)c12]

17. (canceled)

18. A pharmaceutical composition comprising an antibody-drug conjugate according to claim 1, and a pharmaceutically acceptable excipient.

19. (canceled)

20. A method of treating an HIV infection in a subject comprising administering to the subject an antibody-drug conjugate according to claim 1.

21. A method of treating an HIV-infection in a subject comprising administering to the subject a pharmaceutical formulation according to claim 18.

22. An antibody-drug conjugate of Formula (I):
Ab-[L-Dₙ]ₓ (I)
wherein:
Ab comprises a broadly neutralizing anti-HIV antibody;
L comprises a linker molecule covalently bonded to said broadly neutralizing anti-HIV antibody;
D comprises one or more drugs comprising an HIV therapeutic compound covalently bonded to said linker molecule L, wherein said one or more broadly neutralizing anti-HIV antibodies Ab specifically bind to an HIV envelope glycoprotein and said one or more drugs D specifically bind to an HIV envelope glycoprotein;
n is selected from 1-4; and
x is selected from 1-12.

23. The antibody-drug conjugate according to claim 22, wherein the broadly neutralizing antibody Ab binds to the HIV envelope glycoprotein selected from the group consisting of gp160, gp120 and gp41.

24.-25. (canceled)

26. The antibody-drug conjugate according to claim 22, wherein the broadly neutralizing antibody Ab binds to the HIV envelope glycoprotein at the gp120/gp41-interface, at the CD4-binding site, or to the gp41 membrane-proximal external region (MPER).

27.-46. (canceled)

47. The antibody-drug conjugate according to claim 22, wherein the linker molecule is a non-cleavable linker.

48.-49. (canceled)

50. The antibody-drug conjugate according to claim 22, wherein the drug D specifically binds to a HIV envelope glycoprotein selected from the group consisting of gp160, gp120 and gp41.

51. The antibody-drug conjugate according to claim 22, wherein the drug D is an attachment inhibitor.

52.-53. (canceled)

54. The antibody-drug conjugate according to claim 22, wherein the drug D is a compound of the formula:

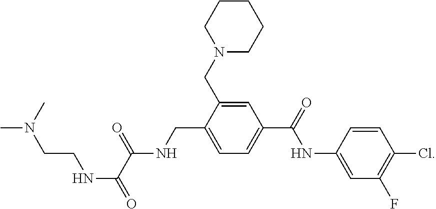 [SMILES: CN(C)CCNC(=O)C(=O)NCc1ccc(C(=O)Nc2ccc(Cl)c(F)c2)cc1CN1CCCCC1]

55. The antibody-drug conjugate according to claim 22, wherein the drug D is a compound of the formula:

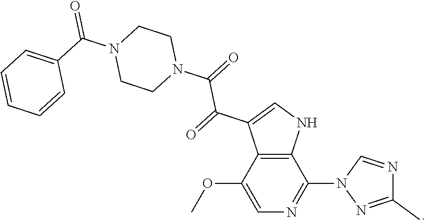 [SMILES: COc1cnc(-n2cnc(C)n2)c2[nH]cc(C(=O)C(=O)N3CCN(C(=O)c4ccccc4)CC3)c12]

56. The antibody-drug conjugate according to claim 22, wherein the drug D is a peptide which binds to CD4.

57.-58. (canceled)

59. The antibody-drug conjugate according to claim 22, wherein the drug D is of the formula A: 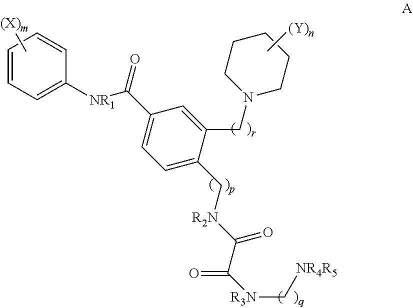
wherein:
X and Y are independently selected from the group consisting of H, (C₁-C₆)alkyl, (C₁-C₆)alkoxy, halo, oxo, haloalkyl, bihaloalkyl, trihaloalkyl, haloalkoxy, bihaloalkoxy, trihaloalkoxy, hydroxyl, amino, amide and (C₁-C₆)alkyl-(C═O);
R₁, R₂, R₃, R₄ and R₅ are each independently selected from H or (C₁-C₆)alkyl;
m ranges from 0 to 5;
n ranges from 0 to 5;
r ranges from 1 to 6;
p ranges from 1 to 6; and
q ranges from 1 to 6.

60. The antibody-drug conjugate according to claim 59, wherein:
X is selected from Cl and F;
Y is H;
m is 2;
n is 1;
R₁, R₂, R₃, R₄ and R₅ are each independently H;
r ranges from 1 to 4;
p ranges from 1 to 4; and
q ranges from 1 to 4.

61. (canceled)

62. An antibody-drug conjugate of Formula (I):
Ab-[L-Dₙ]ₓ (I)
wherein:
Ab comprises a broadly neutralizing anti-HIV antibody;
L comprises a linker molecule covalently bonded to said broadly neutralizing anti-HIV antibody;
D comprises one or more drugs comprising an HIV therapeutic compound covalently bonded to said linker molecule L, wherein said one or more broadly neutralizing anti-HIV antibodies Ab specifically bind to an HIV envelope glycoprotein and said one or more drugs D specifically bind to an HIV envelope glycoprotein;
n is selected from 1-4;
x is selected from 1-12, wherein the antibody-drug-conjugate comprises (1) a first drug D covalently bonded to a first linker molecule L, which is covalently bonded to said broadly neutralizing antibody and (2) a second drug D covalently bonded to a second linker molecule L, which is covalently bonded to said broadly neutralizing antibody.

63. The antibody-drug conjugate according to claim 62, wherein the first drug D is the same as the second drug D.

64. The antibody-drug conjugate according to claim 62, wherein the first drug D is different than the second drug D.

65. The antibody-drug-conjugate according to claim 62, wherein the two drugs are selected from the group consisting of gp120 attachment inhibitors, gp160 attachment inhibitors and combinations thereof.

66. (canceled)

67. A pharmaceutical composition comprising an antibody-drug conjugate according to claim 22, and a pharmaceutically acceptable excipient.

68. The pharmaceutical composition according to claim 67, comprising one or more additional HIV therapeutic agents.

69. A method of treating, curing or preventing an HIV infection in a subject comprising administering to the subject an antibody-drug conjugate according to claim 22.

70. A method of treating, curing or preventing an HIV-infection in a subject comprising administering to the subject a pharmaceutical composition according to claim 67.

71.-77. (canceled)


SEQUENCE LISTING (27 sequences)

SEQ ID NO: 1 - protein, 345 aa, Artificial Sequence
  Synthetic polypeptide
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 36 aa, Artificial Sequence
  Synthetic polypeptide
YTSLIHSLIEESQNQQEKNEQELLELDKWASLWNWF

SEQ ID NO: 3 - protein, 6 aa, Artificial Sequence
  Synthetic peptide
DCTLNW

SEQ ID NO: 4 - protein, 16 aa, Artificial Sequence
  Synthetic peptide
LKPRGGAVNYARPLQG

SEQ ID NO: 5 - protein, 12 aa, Artificial Sequence
  Synthetic peptide
GKNCDYNWDFEH

SEQ ID NO: 6 - protein, 9 aa, Artificial Sequence
  Synthetic peptide
RTSQYGSLA

SEQ ID NO: 7 - protein, 7 aa, Artificial Sequence
  Synthetic peptide
SGSTRAA

SEQ ID NO: 8 - protein, 5 aa, Artificial Sequence
  Synthetic peptide
QQYEF

SEQ ID NO: 9 - protein, 120 aa, Artificial Sequence
  Synthetic polypeptide
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVS

SEQ ID NO: 10 - protein, 105 aa, Artificial Sequence
  Synthetic polypeptide
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRT

SEQ ID NO: 11 - protein, 451 aa, Artificial Sequence
  Synthetic polypeptide
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 451 aa, Artificial Sequence
  Synthetic polypeptide
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 13 - protein, 104 aa, Artificial Sequence
  Synthetic polypeptide
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKR

SEQ ID NO: 14 - protein, 5 aa, Artificial Sequence
  Synthetic peptide
AHILF

SEQ ID NO: 15 - protein, 17 aa, Artificial Sequence
  Synthetic peptide
WIKPQYGAVNFGGGFRD

SEQ ID NO: 16 - protein, 13 aa, Artificial Sequence
  Synthetic peptide
DRSYGDSSWALDA

SEQ ID NO: 17 - protein, 11 aa, Artificial Sequence
  Synthetic peptide
QTSQGVGSDLH

SEQ ID NO: 18 - protein, 7 aa, Artificial Sequence
  Synthetic peptide
HTSSVED

SEQ ID NO: 19 - protein, 5 aa, Artificial Sequence
  Synthetic peptide
QVLQF

SEQ ID NO: 20 - protein, 122 aa, Artificial Sequence [sequence omitted]

SEQ ID NO: 21 - protein, 103 aa, Artificial Sequence [sequence omitted]

SEQ ID NO: 22 - protein, 452 aa, Artificial Sequence
  Synthetic polypeptide
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 210 aa, Artificial Sequence
  Synthetic polypeptide
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 452 aa, Artificial Sequence
  Synthetic polypeptide
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 25 - protein, 479 aa, Artificial Sequence
  Synthetic polypeptide
KLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 26 - protein, 125 aa, Artificial Sequence
  Synthetic polypeptide
QVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSS

SEQ ID NO: 27 - protein, 101 aa, Artificial Sequence
  Synthetic polypeptide
SLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK